(19) United States
(12) Patent Application Publication
(10) Pub. No.: US 2023/0335289 A1
(43) Pub. Date: Oct. 19, 2023

(54) SYSTEMS AND METHODS FOR GENERATING HEALTH RISK ASSESSMENTS

(71) Applicant: A-Rhythmik GmbH, Hamburg (DE)

(72) Inventor: Karl-Heinz KUCK, Hamburg (DE)

(21) Appl. No.: 18/106,794

(22) Filed: Feb. 7, 2023

Related U.S. Application Data
(63) Continuation of application No. PCT/EP2021/072204, filed on Aug. 9, 2021.
(60) Provisional application No. 63/063,705, filed on Aug. 10, 2020.
(60) Provisional application No. 63/156,671, filed on Mar. 4, 2021.

(51) Int. Cl. G16H 50/30; G16H 40/67; G16H 50/20; G16H 10/60

(52) U.S. Cl. CPC G16H 50/30; G16H 40/67; G16H 50/20; G16H 10/60

(57) ABSTRACT

Provided herein are systems and methods for assessing health risk for a user from a combination of Artificial Intelligence, ECG data available from the user's smart watch or other smart device, and other biometric data and/or medical data provided from the user.

### CROSS-REFERENCE

This application is a continuation of PCT Application No. PCT/EP2021/072204, filed Aug. 9, 2021; which claims the benefit of U.S. Provisional Application No. 63/063,705, filed Aug. 10, 2020; and U.S. Provisional Application No. 63/156,671, filed on Mar. 4, 2021; each of which is incorporated herein by reference in its entirety.

### BACKGROUND

The present disclosure is related to medical diagnostic systems, devices, and methods, particularly for electrocardiogram (ECG) and other bioelectrical signals.

Electrocardiogram (ECG) readings are used to evaluate a patient's heart and detect signs of heart disease. Typical ECGs are conducted using a 12-lead ECG device. Smart devices, such as a smart watch, can also be configured to act as an ECG device, wherein the smart watch may include a software application that receives and outputs the ECG readings.

### SUMMARY

The present disclosure generally relates to systems and methods for generating health risk assessments, which may comprise diagnosing cardiac arrhythmia or otherwise process and glean information from cardiac electrical signals, and more particularly relates to using Artificial Intelligence (AI) to improve identification of irregularities with ECG data and determine health risks based on a user's specific characteristics.

Disclosed herein, in some embodiments, is a method for generating a health risk assessment for a user, comprising: a) providing biometric information and/or medical information relating to the user; b) receiving electrocardiogram (ECG) data measured from the user; c) evaluating the ECG data by applying a machine learning algorithm to characterize the ECG data according to a heart rhythm; and d) generating the health risk assessment based on the biometric information, the medical information, the evaluated ECG data, or a combination thereof.

In some embodiments, the heart rhythm characterizations comprise sinus rhythm or a type of cardiac arrhythmia. In some embodiments, the cardiac arrhythmia comprises one or more of atrial fibrillation, atrial extrasystole, nonsustained ventricular tachycardia, atrial flutter, atrial tachycardia, pacemaker ECG, atrioventricular (AV) nodal reentrant tachycardia, AV reentry tachycardia (Wolff-Parkinson-White syndrome) and ventricular tachycardia, or sinoatrial conduction block and AV conduction block.

In some embodiments, the generating of the health risk assessment comprises computing a cardiovascular risk age.

In some embodiments, the method further comprising: a) receiving a plurality of sets of ECG data measured from the user over a plurality of temporal intervals; and b) classifying the user according to a heart rhythm category based on the frequency of irregularities identified from the plurality of sets of ECG data.

In some embodiments, the generating of the health risk assessment comprises calculating a stroke risk based on the heart rhythm category for the user.

In some embodiments, the method further comprises providing the user with recommended actions based on the determined stroke risk. In some embodiments, the generating of the health risk assessment comprises determining a sudden cardiac death (SCD) risk based on the heart rhythm category for the user. In some embodiments, the SCD risk is determined based on the type of cardiac arrhythmia identified. In some embodiments, the SCD risk is determined based on the presence and/or absence of fractionated QRS complexes identified with the ECG data.

In some embodiments, the biometric information and/or medical information comprises one or more of height, weight, waist circumference, smoking status, country of residence, total cholesterol, LDL cholesterol, systolic blood pressure, medication, congestive heart failure, ischemic cardiomyopathy, nonischemic cardiomyopathy, NYHA functional class, LV ejection fraction, hypertension, diabetes mellitus, glycated hemoglobin (HbA1c), vascular disease, prior TIA/stroke/thromboembolism, prior myocardial infarction, valvular heart disease, pacemaker, syncope, or a combination thereof.

In some embodiments, the method further comprising receiving one or more symptoms relating to the user, wherein the generated health risk assessment is based at least in part on the one or more symptoms. In some embodiments, the one or more symptoms comprises one or more of chest pain, palpitations, rapid heart rate, dyspnea, dizziness, presyncope, syncope, sweating, speech disorder, weakness/paralysis, or a combination thereof.

In some embodiments, the health risk assessment comprises one or more of a cardiovascular age, a stroke risk, a sudden cardiac death risk, or a combination thereof.

In some embodiments, the evaluating the ECG data comprises: a) creating a graphical output based on the ECG data; b) determining a statistical computation corresponding to the graphical output; and c) comparing the statistical computation with a reference statistical data so as to i) identify ECG data corresponding to sinus rhythm, ii) identify ECG data corresponding to a type of cardiac arrhythmia, or iii) a combination thereof. In some embodiments, the type of cardiac arrhythmia comprises atrial fibrillation. In some embodiments, the graphical output comprises a column chart of R-R intervals from the ECG data. In some embodiments, the statistical computation comprises 1) mean and standard deviation of the R-R intervals, 2) the median (e.g., 50th percentile) of the R-R intervals, 3) 25th and 75th percentile of the R-R intervals, 4) root mean square of successive differences between R-R intervals, or 5) any combination thereof. In some embodiments, the type of cardiac arrhythmia comprises atrial extrasystoles or ventricular extrasystoles. In some embodiments, the type of cardiac arrhythmia is identified based on the graphical output and a computed QRS complex duration. In some embodiments, the reference statistical data corresponds to a plurality of sets of reference ECG data, wherein the plurality of sets of reference ECG data is obtained from a plurality of other users, wherein each set of reference ECG data of the plurality of set of reference ECG data comprises reference R-R intervals corresponding to sinus rhythm and a corresponding mean heart rate range, wherein each set of reference ECG data comprises at least 50, 100, 200, 500, or 1000 electrocardiograms corresponding to sinus rhythm. In some embodiments, the statistical computation is compared with a corresponding set of reference ECG data of the reference statistical data with reference to 1) a specified range about a mean of the reference R-R intervals from the corresponding set of reference ECG data, 2) a specified range for the median of the reference R-R intervals from the corresponding set of reference ECG data, 3) a specified range for the root mean square of successive differences between R-R intervals from the corresponding set of reference ECG data, or any combination thereof.

In some embodiments, the evaluating the ECG data comprises: a) creating a graphical output based on the ECG data; and b) identifying the ECG data as corresponding to sinus rhythm, atrial fibrillation, atrial extrasystole, or ventricular extrasystole based on the graphical output.

In some embodiments, the method further comprises providing a computer readable medium or media encoding instructions that define the machine learning algorithm.

In some embodiments, the method further comprises providing a processor, and a memory coupled to the processor and storing instructions for the processor, to generate the health risk assessment.

Disclosed herein, in some embodiments, is a computer-implemented system comprising: a processor, a memory coupled to the processor and storing instructions for the processor to generate a health risk assessment application comprising: a) a database comprising: i) biometric information and/or medical information for a user; ii) one or more of sinus rhythm data sets corresponding to normal sinus rhythm of a heart; iii) one or more arrhythmia data sets corresponding to one or more types of cardiac arrhythmias; and iv) past ECG data received from a ECG device and measured from the user; b) a software module receiving ECG data measured from the user by an ECG device; c) a software module evaluating the received ECG data using a machine learning algorithm, the one or more sinus rhythm data sets, the one or more arrhythmia data sets, or a combination thereof, so as to characterize the ECG data according to a type of heart rhythm; and d) a software module generating a health risk assessment based on the biometric information, the medical information, the type of heart rhythm, or a combination thereof.

In some embodiments, the heart rhythm characterization comprises sinus rhythm or a type of cardiac arrhythmia. In some embodiments, the cardiac arrhythmia comprises one or more of atrial fibrillation, atrial extrasystole, nonsustained ventricular tachycardia, atrial flutter, atrial tachycardia, pacemaker ECG, atrioventricular (AV) nodal reentrant tachycardia, AV reentry tachycardia (Wolff-Parkinson-White syndrome) and ventricular tachycardia, or sinoatrial conduction block and AV conduction block.

In some embodiments, the received ECG data and type of heart rhythm are received by said database. In some embodiments, generating the health risk assessment comprises computing a cardiovascular risk age.

In some embodiments, the system further comprises a classification module configured to classify the user according to a heart rhythm category based on the heart rhythm characterization and the past ECG data. In some embodiments, the classification module generates an ECG schedule for the user based on the heart rhythm category, wherein the ECG schedule identifies the frequency that ECG data is to be measured from the user. In some embodiments, generating the health risk assessment comprises determining a stroke risk based on the heart rhythm category for the user. In some embodiments, the system further comprises providing the user with recommended actions based on the determined stroke risk.

In some embodiments, generating the health risk assessment comprises determining a sudden cardiac death (SCD) risk based on the heart rhythm category for the user. In some embodiments, the SCD risk is determined based on the type of cardiac arrhythmia identified. In some embodiments, the SCD risk is determined based on the presence and/or absence of fractionated QRS complexes identified with the ECG data. In some embodiments, the biometric information and/or medical information comprises one or more of height, weight, waist circumference, smoking status, country of residence, total cholesterol, LDL cholesterol, systolic blood pressure, medication, congestive heart failure, ischemic cardiomyopathy, nonischemic cardiomyopathy, NYHA functional class, LV ejection fraction, hypertension, diabetes mellitus, glycated hemoglobin (HbA1c), vascular disease, prior TIA/stroke/thromboembolism, prior myocardial infarction, valvular heart disease, pacemaker, syncope, or combinations thereof.

In some embodiments, the system further comprises a software module configured to receive one or more symptoms from the user, wherein the generated health risk assessment is based at least in part on the. one or more symptoms. In some embodiments, the one or more symptoms comprises one or more of chest pain, palpitations, rapid heart rate, dyspnea, dizziness, presyncope, syncope, sweating, speech disorder, weakness/paralysis, or combinations thereof.

In some embodiments, the health risk assessment comprises one or more of a cardiovascular age, a stroke risk, a sudden cardiac death risk, or combinations thereof.

In some embodiments, the evaluating the ECG data comprises: a) creating a graphical output based on the ECG data; b) determining a statistical computation corresponding to the graphical output; and c) comparing the statistical computation and a reference statistical data so as to identify ECG data corresponding to sinus rhythm, identify ECG data corresponding to a type of cardiac arrhythmia, or a combination thereof. In some embodiments, the type of cardiac arrhythmia comprises atrial fibrillation. In some embodiments, the graphical output comprises a column chart of R-R intervals from the ECG data. In some embodiments, the statistical computation comprises 1) mean and standard deviation of the R-R intervals, 2) the median (e.g., 50th percentile) of the R-R intervals, 3) 25th and 75th percentile of the R-R intervals, 4) root mean square of successive differences between R-R intervals, or 5) any combination thereof. In some embodiments, the type of cardiac arrhythmia comprises atrial extrasystoles or ventricular extrasystoles and is identified based on the graphical output and a computed QRS complex. In some embodiments, the reference statistical data corresponds to a plurality of sets of reference ECG data, wherein the plurality of sets of reference ECG data is obtained from a plurality of other users, wherein each set of reference ECG data of the plurality of set of reference ECG data comprises reference R-R intervals corresponding to sinus rhythm and a corresponding mean heart rate range, wherein each set of reference ECG data comprises at least 50, 100, 200, 500, or 1000 electrocardiograms corresponding to sinus rhythm. In some embodiments, the statistical computation is compared with a corresponding set of reference ECG data of the reference statistical data with reference to i) a specified range about a mean of the reference R-R intervals from the corresponding set of reference ECG data, ii a specified range for the median of the reference R-R intervals from the corresponding set of reference ECG data, iii) a specified range for the root mean square of successive differences between R-R intervals from the corresponding set of reference ECG data, or any combination thereof.

In some embodiments, the evaluating the ECG data comprises: a) creating a graphical output based on the ECG data; and b) identifying the ECG data as corresponding to sinus rhythm, atrial fibrillation, atrial extrasystole, or ventricular extrasystole based on the graphical output.

Disclosed herein, in some embodiments, is a method for predicting a stroke risk for a user, comprising: a) receiving biometric information and/or medical information from the user; b) providing a plurality of sets of electrocardiogram (ECG) data measured from the user using an ECG device, wherein the plurality of sets of ECG data is measured over a plurality of temporal intervals; c) identifying i) ECG data corresponding to sinus rhythm, ii) irregularities with the plurality of sets of ECG data, or iii) both; d) classifying the user according to a heart rhythm category based on the frequency of irregularities identified; and e) determining a stroke risk for the user based on the heart rhythm category for the user. In some embodiments, the method further comprises providing the user with a recommended action based on the determined stroke risk. In some embodiments, the recommended action comprise a frequency for measuring one or more additional ECG data from the user.

Disclosed herein, in some embodiments, is a method for predicting a sudden cardiac death (SCD) risk for a user, comprising: a) receiving biometric information and/or medical information from the user; b) providing a plurality of sets of electrocardiogram (ECG) data measured from the user using an ECG device, wherein the plurality of sets of ECG data is measured over a plurality of temporal intervals; c) identifying i) ECG data corresponding to sinus rhythm, ii) irregularities with the plurality of sets of ECG data, or iii) both; d) classifying the user according to a heart rhythm category based on the frequency of irregularities identified; and e) determining a SCD risk for the user based on the heart rhythm category for the user. In some embodiments, the method further comprises identifying a type of cardiac arrhythmia based on the identified irregularities with the plurality of sets of ECG data. In some embodiments, the SCD risk is determined based on the type of cardiac arrhythmia(s) identified. In some embodiments, the cardiac arrhythmia comprises one or more of atrial fibrillation, atrial extrasystole, nonsustained ventricular tachycardia, atrial flutter, atrial tachycardia, pacemaker ECG, atrioventricular (AV) nodal reentrant tachycardia, AV reentry tachycardia (Wolff-Parkinson-White syndrome) and ventricular tachycardia, or sinoatrial conduction block and AV conduction block. In some embodiments, the sudden cardiac death risk is determined based on the presence and/or absence of fractionated QRS complexes identified with the ECG data.

Disclosed herein, in some embodiments, is a computer-implemented method for training a network for cardiac arrhythmia detection, comprising: a) collecting one or more sets of arrhythmia electrocardiogram (ECG) data corresponding to i) one or more types of cardiac arrhythmia, and ii) one or more sets of sinus rhythm ECG data corresponding to normal sinus rhythm of a heart; b) generating detection criteria for each type of cardiac arrhythmia of the one or more types of cardiac arrhythmia based on i) the one or more sets of arrhythmia ECG data, ii) the one or more sets of sinus rhythm ECG data, or iii) both, wherein each detection criteria is generated using a machine learning algorithm, wherein each detection criteria is based on specific ECG data parameters that correspond with a respective cardiac arrhythmia type; c) receiving a new data set corresponding to a type of cardiac arrhythmia of the one or more types of cardiac arrhythmia; and d) adjusting the detection criteria for the type of cardiac arrhythmia based on the new data set using the machine learning algorithm. In some embodiments, the method further comprises identifying a type of cardiac arrythmia associated with raw ECG data measured from a user, wherein said identifying is based on matching the raw ECG data with the specific ECG data parameters of a detection criteria for a type of cardiac arrhythmia. In some embodiments, the detection criteria for each type of cardiac arrhythmia is generated based on at least 100 corresponding data sets. In some embodiments, the detection criteria for each type of cardiac arrhythmia is generated based on at least 1,000 corresponding data sets. In some embodiments, the detection criteria for each type of cardiac arrhythmia is generated based on at least 10,000 corresponding data sets. In some embodiments, the detection criteria for each type of cardiac arrhythmia is generated based on at least 20,000 corresponding data sets.

Disclosed herein, in some embodiments, is a computer-implemented system comprising: a processor, and a memory coupled to the processor and storing instructions for the processor generate a cardiac arrhythmia determination application for identifying cardiac arrhythmia comprising: a) a database comprising one or more sets of electrocardiogram (ECG) data corresponding to one or more types of cardiac arrhythmia; b) a detection module configured to generate detection criteria for each type of cardiac arrhythmia of the one or more types of cardiac arrhythmia using a machine learning algorithm, wherein each detection criteria is based on specific ECG data parameters that correspond with a respective type of cardiac arrhythmia; c) a receiving module configured to receive a new data set corresponding to a type of cardiac arrhythmia of the one or more types of cardiac arrhythmia; wherein the detection module uses the machine learning algorithm to adjust the detection criteria for the type of cardiac arrhythmia corresponding to the new data set, wherein said adjustment is based on the new data set; and d) an identification module configured to associate raw ECG data measured from a user with a type of cardiac arrhythmia, wherein the raw ECG data is matched with the specific ECG data parameters of a detection criteria for a type of cardiac arrhythmia. In some embodiments, the detection criteria for each type of cardiac arrhythmia is generated based on at least 100 corresponding data sets. In some embodiments, the detection criteria for each type of cardiac arrhythmia is generated based on at least 1,000 corresponding data sets. In some embodiments, the detection criteria for each type of cardiac arrhythmia is generated based on at least 10,000 corresponding data sets. In some embodiments, the detection criteria for each type of cardiac arrhythmia is generated based on at least 20,000 corresponding data sets.

Disclosed herein, in some embodiments, is a method for characterizing a health condition for a user, comprising: a) receiving electrocardiogram (ECG) data that is measured from the user; b) creating a graphical output based on the ECG data; c) determining a statistical computation corresponding to the graphical output; and d) characterizing the health condition based on a comparison between the statistical computation and a reference statistical data. In some embodiments, the graphical output comprises a column chart of R-R intervals from the ECG data. In some embodiments, the statistical computation comprises 1) mean and standard deviation of the R-R intervals, 2) the median (e.g., 50th percentile) of the R-R intervals, 3) 25th and 75th percentile of the R-R intervals, 4) root mean square of successive differences between R-R intervals, or 5) any combination thereof. In some embodiments, the health condition comprises sinus rhythm or atrial fibrillation. In some embodiments, the method further comprises identifying atrial extrasystoles or ventricular extrasystoles based on the graphical output and a computed QRS complex. In some embodiments, the reference statistical data corresponds to a plurality of sets of reference ECG data, wherein the plurality of sets of reference ECG data is obtained from a plurality of other users, wherein each set of reference ECG data of the plurality of set of reference ECG data comprises reference R-R intervals corresponding to sinus rhythm and a corresponding mean heart rate range, wherein each set of reference ECG data comprises at least 50, 100, 200, 500, or 1000 electrocardiograms corresponding to sinus rhythm. In some embodiments, the statistical computation is compared with a corresponding set of reference ECG data with reference to 1) a specified range about a mean of the reference R-R intervals from the corresponding set of reference ECG data, 2) a specified range for the median of the reference R-R intervals from the corresponding set of reference ECG data, 3) a specified range for the root mean square of successive differences between R-R intervals from the corresponding set of reference ECG data, or any combination thereof.

Disclosed herein, in some embodiments, is a method for characterizing a health condition for a user, comprising: a) receiving electrocardiogram (ECG) data that is measured from the user; b) creating a graphical output based on the ECG data; and c) characterizing the health condition based on the graphical output. In some embodiments, the characterizing comprises identifying the ECG data as corresponding to sinus rhythm, atrial fibrillation, atrial extrasystole, or ventricular extrasystole. In some embodiments, the characterizing comprises identifying the ECG data as corresponding to any type of cardiac arrhythmia described herein.

### BRIEF DESCRIPTION OF THE DRAWINGS

Specific embodiments of the disclosed devices, delivery systems, or methods will now be described with reference to the drawings. Nothing in this detailed description is intended to imply that any particular component, feature, or step is essential to the invention.

FIG. 1 depicts an exemplary illustration of a system described herein.

FIG. 2 depicts an exemplary illustration of another system described herein.

FIG. 3A depicts exemplary ECG data corresponding to sinus rhythm.

FIG. 3B depicts a R-R interval column chart based on the ECG data from FIG. 3A.

FIG. 4A depicts a second exemplary ECG data corresponding to sinus rhythm.

FIG. 4B depicts a R-R interval column chart based on the ECG data from FIG. 4A.

FIG. 5A depicts exemplary ECG data corresponding to atrial fibrillation.

FIG. 5B depicts a R-R interval column chart based on the ECG data from FIG. 5A.

FIG. 6A depicts exemplary ECG data corresponding to sinus rhythm with Atrial extrasystole (AES).

FIG. 6B depicts a R-R interval column chart based on the ECG data from FIG. 6A.

FIG. 7A depicts exemplary ECG data corresponding to sinus rhythm with Ventricular extrasystole (VES).

FIG. 7B depicts a R-R interval column chart based on the ECG data from FIG. 7A.

FIG. 8 depicts a flowchart for an exemplary process for evaluating an unknown ECG data.

FIG. 9 depicts an exemplary chart for determining cardiovascular health risk for low-risk populations.

FIG. 10 depicts an exemplary chart for high-risk populations.

FIG. 11 depicts a flow chart for exemplary method described herein for generating a health risk assessment.

FIG. 12 illustrates a schematic of a computer system that is programmed or otherwise configured to implement methods provided herein.

FIG. 13A depicts an exemplary graphical output of ECG data (ECG recording) for sinus rhythm.

FIG. 13B depicts an exemplary graphical output of a secondary analysis of the ECG data from FIG. 13A.

FIG. 13C depicts an exemplary R-R interval graph based on the ECG data from FIG. 13A.

FIG. 13D depicts exemplary secondary characteristics of the ECG data based on the output from FIG. 13B.

FIG. 14A depicts an exemplary graphical output of ECG data (ECG recording) for sinus rhythm with ventricular extrasystoles.

FIG. 14B depicts an exemplary graphical output of a secondary analysis of the ECG data from FIG. 14A.

FIG. 14C depicts an exemplary R-R interval graph based on the ECG data from FIG. 14A.

FIG. 14D depicts exemplary secondary characteristics of the ECG data based on the output from FIG. 14B.

FIG. 15A depicts an exemplary graphical output of ECG data (ECG recording) for sinus rhythm with atrial extrasystoles.

FIG. 15B depicts an exemplary graphical output of a secondary analysis of the ECG data from FIG. 15A.

FIG. 15C depicts an exemplary R-R interval graph based on the ECG data from FIG. 15A.

FIG. 15D depicts exemplary secondary characteristics of the ECG data based on the output from FIG. 15B.

FIG. 16A depicts examples of Narrow-QRS Fragmentation.

FIG. 16B depicts examples of wide QRS complexes.

### DETAILED DESCRIPTION

Disclosed herein are systems and methods for generating health risk assessments that may be based on ECG data measured from a user. The systems and methods may use Artificial Intelligence (AI) to characterize any cardiac arrhythmia on a 1-channel ECG better than a general cardiologist (such as shown with a clinical superiority trial). The machine learning algorithms associated with the AI may provide a significant improvement over the existing art. The terms “AI” and “artificial intelligence” may be used interchangeably with the term “classifier” herein. The health risk assessment generating systems and methods disclosed herein provide a user with an arrhythmia detection software application (“AD app”) that may be in communication with an Electrocardiogram (ECG) device, wherein the AD app may be configured to read ECG raw data measured from a user by the ECG device. The ECG device may measure and record the ECG raw data from lead I of a regular 12-lead ECG or a wearable device, such as a smartwatch. The smartwatch may include only one lead which may correspond to lead I of a 12-lead ECG. The measured ECG raw data from a regular 12-lead ECG or wearable device (e.g., smartwatch) will then be transmitted to a cloud network. The AD app may be configured on a computing device, such as a smartphone, tablet, wearable computing device (e.g., smartwatch, which may be the same as the ECG device as described herein), computer, or combinations thereof. The AD app may be an “app” that is downloaded from an “app store”, such as the “App Store” configured for Apple iOS computing devices (e.g., iPhone, Apple Watch, etc.), the “Google Play” app store configured for Google Android computing devices (e.g., Samsung Galaxy series of smartphones and smartwatches, Google Pixel series of smart phones), or the like.

The ECG device may be coupled to a wearable device. The ECG device may be configured as part of a wearable device. The wearable device may be a smart device, such as a smart watch (e.g., an Apple watch, a Samsung Galaxy Watch, a FitBit Versa, and the like). The smart watch and/or corresponding smart phone, tablet, computer or combinations thereof (e.g., Apple watch corresponding with an Apple IPHONE, Apple IPAD, Apple MacBook, or combinations thereof, or a Samsung Galaxy Watch, a FitBit Versa, or other Android-based smartwatch corresponding with a Samsung Galaxy smartphone, tablet computer, or other Android-based computing device) may include a software application (“ECG app”) that is in communication with the ECG device, such that the ECG app is configured to receive raw ECG data measured by the ECG device. The ECG app may be an “app” configured with the smart watch (and corresponding smartphone/table, computer, etc), such as the Apple Health app, and/or the ECG app may be an “app” downloaded from an “app store”.

The AD app may be in communication with the ECG app such that the AD app reads the ECG raw data obtained through the ECG app. The AD app may be configured to provide information on a display of a computing device, such as a smartphone, smartwatch, tablet. The AD app may be configured to receive feedback from a user. Via the AD app, non-personal information and symptoms of a user may be collected so as to assess the personal health risks of the user. Collected non-personal information may comprise biometric data and/or medical data, which may comprise one or more of height, weight, waist circumference, smoking status, country of residence, total cholesterol, LDL cholesterol, systolic blood pressure, medication, congestive heart failure, ischemic cardiomyopathy, nonischemic cardiomyopathy, NYHA functional class, LV ejection fraction, hypertension, diabetes mellitus, glycated hemoglobin (HbAlc), vascular disease, prior TIA/stroke, prior myocardial infarction, valvular heart disease, pacemaker, syncope, or combinations thereof. Collected symptoms may comprise chest pain, palpitations, rapid heart rate, dyspnea, dizziness, presyncope, syncope, sweating, speech disorder, weakness/paralysis, or combinations thereof.

The AD app may use Artificial Intelligence (AI), which may comprise a machine learning algorithm, that may be trained to detect the normal sinus rhythm as well as three cardiac arrhythmias: atrial fibrillation, atrial extrasystoles and ventricular extrasystoles. As described herein, the term “AI’ may be used interchangeably with the term “classifier”. A number of trained data sets may be used by the classifier to characterize a received ECG data. For example, at least about 50 trained data sets, about 100 trained data sets, about 500 trained data sets, about 1,000 trained data sets, about 5,000 trained data sets, about 10,000 trained data sets, about 20,000 trained data sets, or about 50,000 trained data sets may be necessary for each diagnosis (e.g., characterization of received ECG data from a user). A trained data set can be used for more than one characterization.

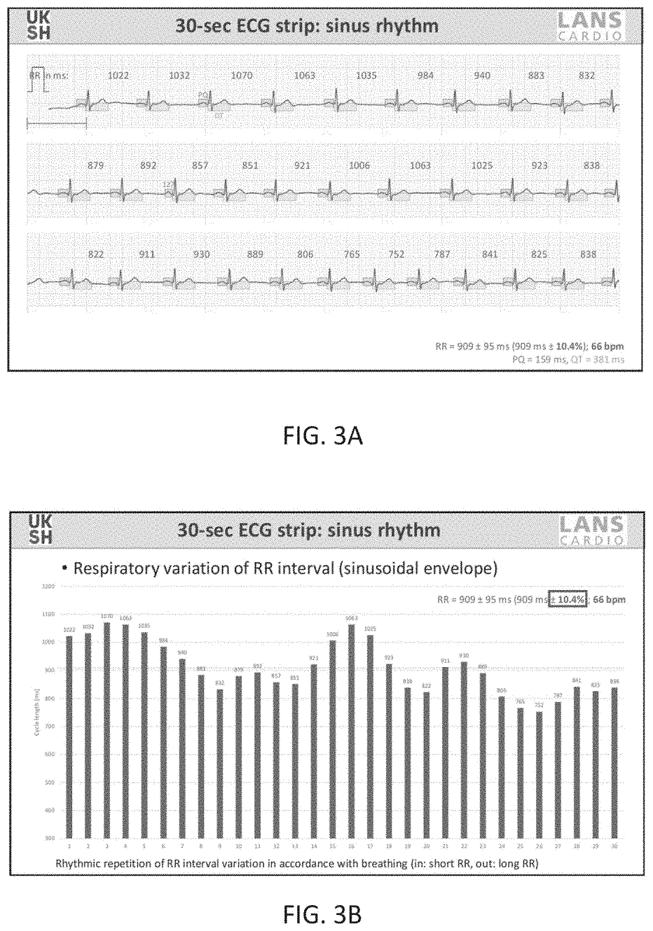

As described herein, the trained data sets may be based at least partially from ECG data based on a plurality of ECG recordings (obtained for example, via an ECG device described herein). Each trained data set may correspond to ECG data from an ECG recording that has been correctly assigned to a specific heart rhythm (e.g., sinus rhythm, atrial fibrillation, any type of cardiac arrhythmia, etc). Each ECG data for each trained data set may be a single channel ECG, which may be obtained from an ECG device (as described herein, for example, a wearable device) and/or via a PDF file (an image of ECG data for example). The trained data sets may include ECG raw data that has been processed (as described herein, for example see FIGS. 3-7, and 13-15). The trained data sets may correspond to ECG data from a plurality of individuals (e.g., 100 trained data sets may correspond to ECG data or readings from up to 100 individuals). Each trained data set may be considered as correctly assigned after being verified by a healthcare professional (e.g., a physician, an electrophysiologist). Accordingly, as described herein, the classifier may be able to receive an uncharacterized ECG data (e.g., ECG data that has not been characterized according to heart rhythm for example), and using the trained data sets, characterized the uncharacterized ECG data (e.g., characterize as sinus rhythm, atrial fibrillation, any type of cardiac arrhythmia, etc). The classifier may be trained to correlate the various permutations of an ECG data characterization (for example, permutations associated with sinus rhythm, or atrial fibrillation). The trained data sets may correspond to a plurality of time-domain data and frequency-domain data.

The machine learning algorithm (as provided with the classifier) may be provided with a computer-implemented system comprising a processor, and a memory, coupled to the processor and for storing instructions for the processor to develop detection criteria for characterizing ECG data. The memory may be configured to store a plurality of sets of ECG data (for example, a plurality of ECG recordings), which have been correctly assigned according to a heart rhythm (e.g., sinus rhythm, a type of cardiac arrhythmia), i.e. a plurality of trained data sets. In some cases, the machine learning algorithm identifies characteristics and parameters associated with the trained ECG data set for each heart rhythm, and may accordingly develop corresponding detection criteria for each heart rhythm type (pertaining to characteristics and parameters of ECG data). As the system receives more ECG data sets that have been correctly assigned, the machine learning algorithm may adjust the detection criteria for a given heart rhythm type. For example, if a plurality of ECG data sets assigned as atrial fibrillation are received and have one or more parameters that are outside of a previous detection criteria range for atrial fibrillation (as determined by the machine learning algorithm), the machine learning algorithm may then adjust the detection criteria for atrial fibrillation to include the range of such one or more parameters.

As described herein, in addition to heart rhythm types such as sinus rhythm, atrial fibrillation, atrial extrasystoles, ventricular extrasystoles, the classifier may be further trained to detect other cardiac arrhythmias such as nonsustained ventricular tachycardia, atrial flutter, atrial tachycardia, pacemaker ECG, atrioventricular (AV) nodal reentrant tachycardia, AV reentry tachycardia (Wolff-Parkinson-White syndrome) and ventricular tachycardia, sinoatrial conduction block and AV conduction block.

With a sufficient amount of ECG data, age, gender and the patient's current symptoms, the classifier may be able to correctly characterize the patient's heart rhythm (either normal sinus rhythm or arrhythmia) and give adequate recommendations for action as described herein, e.g., no action needed, see cardiologist within 24 hours, emergency situation/go to hospital. The recommendations may be provided through the display of the corresponding computing device (as described herein).

By getting more information from the user, the AD app may be able to assess the user's personal health risks. These comprise the risk of experiencing a fatal cardiovascular event (myocardial infarction, stroke, or rupture of an aortic aneurysm) as well as the risks of experiencing a stroke or sudden cardiac death. The AD app may give recommendations, as described herein, based on the personal risks to support the user in improving his/her overall heart health. The recommendations may be provided through the display of the corresponding computing device.

The AD app may be configured to extract data from the ECG app, which includes ECG raw data measured by the ECG device. In addition to processing of ECG raw data, the AD app may be able to process ECGs saved in image files (e.g., PDF reports). Also lead I from a multi-channel ECG may be usable/readable by the classifier. The necessary raw or image data (ECG, cycle length [heart rate], etc.) are exactly defined (e.g., may be raw electronic data corresponding to x- and y-coordinates over time, and/or may be images via for example, PDF data) and the data imported from the ECG app may comply with the General Data Protection Regulation (GDPR) requirements. The AD app may format and/or normalize the ECG data so as to be readable by the classifier and/or to comply with GDPR requirements. The ECG data may be digitized. The ECG data may be digitized by the AD app.

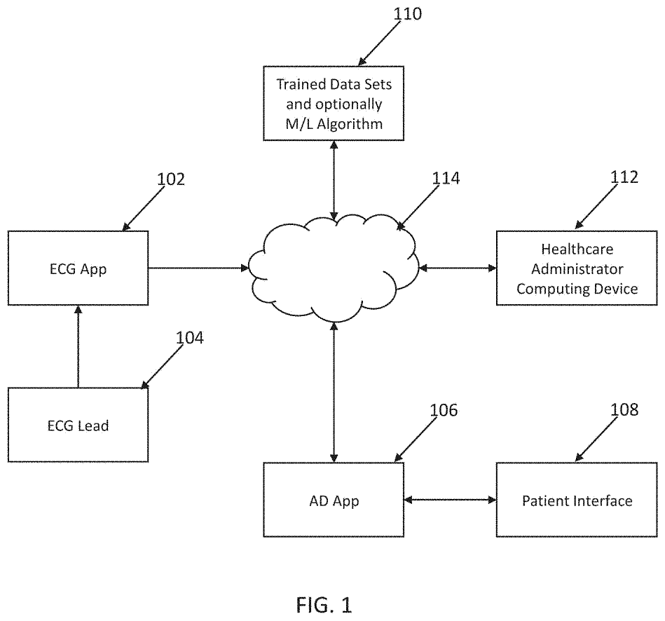

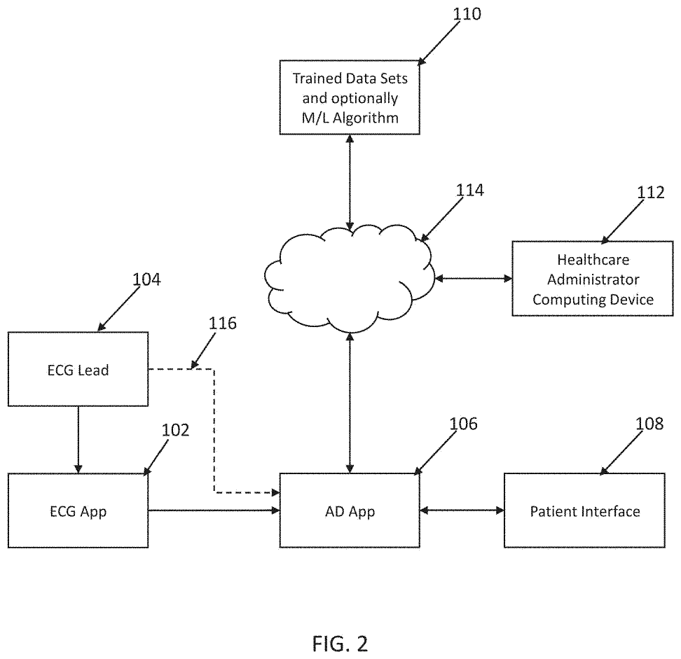

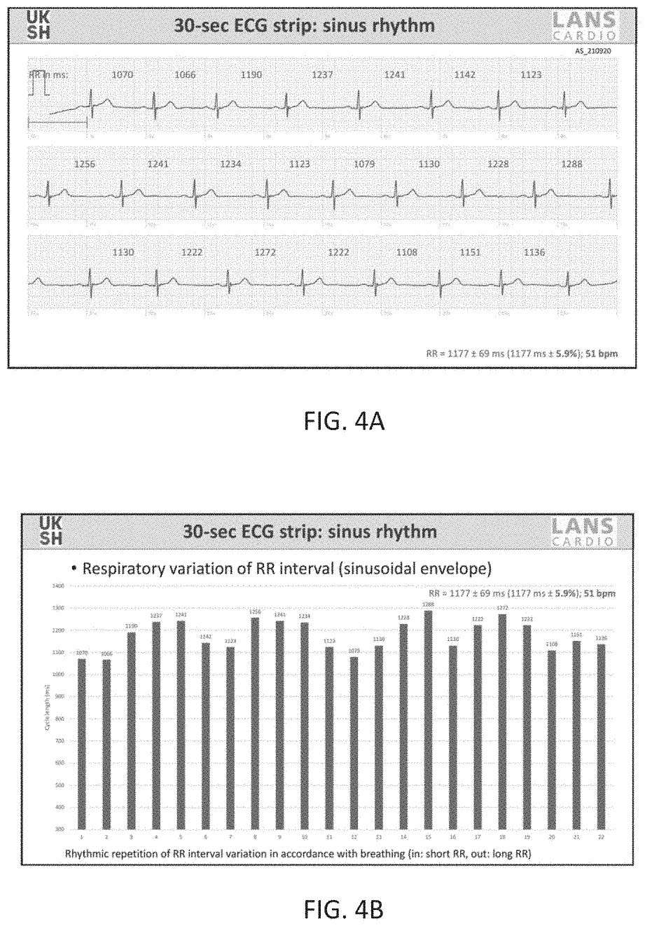

FIGS. 1-2 provide an exemplary depiction of systems described herein, and in particular, the interrelation between some of the system components. FIG. 1 provides an exemplary depiction of a system wherein an ECG device is in communication with a computing device (e.g., a cloud server 114) to provide ECG data measured from a patient. For example, the ECG device may measure and record the ECG raw data (for example, see FIG. 4A) from lead 1 104 of an ECG device (such as a regular 12-lead ECG or a wearable device, such as a smartwatch, as described herein). The ECG device may then communicate the ECG raw data to a computing device, or in some cases, to a software application that is part of a device comprising the ECG device. For example, in cases where a wearable device (e.g., smartwatch) also acts as an ECG device, the wearable device may have an ECG App 102 that receives the measured ECG raw data. The wearable device may then transmit the ECG raw data to another computing device, via for example, a cloud server 114, which may be in communication with other devices. The ECG App 102 may also be able to process the ECG raw data prior to transmitting the data to another computing device (e.g., cloud server).

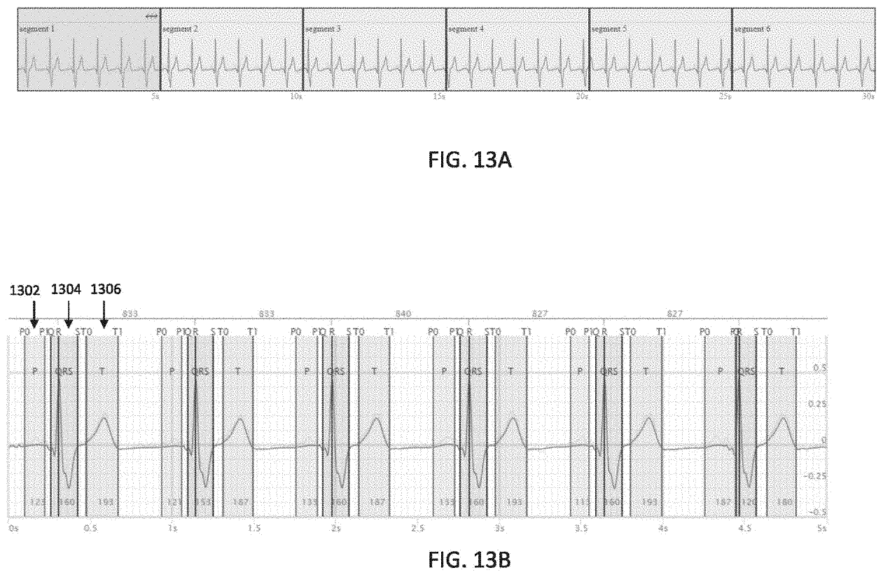

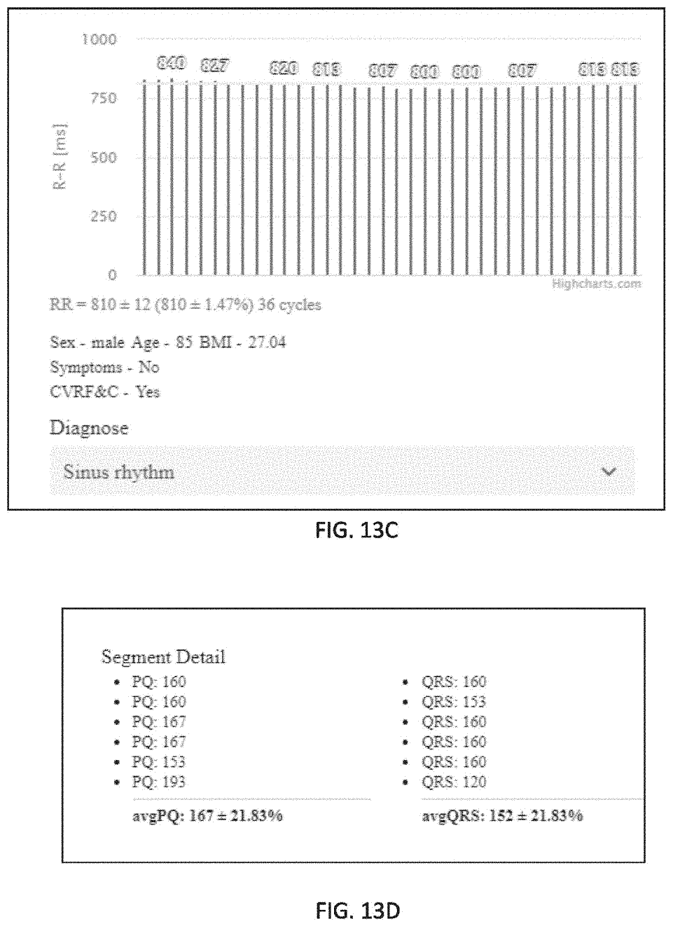

With continued reference to FIG. 1, the AD App 106 may retrieve the ECG data via the other computing device (e.g., via cloud server 114). In some cases, the ECG raw data has already been processed (for e.g., via the ECG App as described herein). In some cases, the AD App 106 retrieves the ECG raw data, and processes it using the AD App 106 for further use (see for example FIGS. 13-15 herein). In some cases, the AD App 106 is configured to send/receive data and/or information from a patient/user via a patient interface 108. For example, the AD App may be provided with a computing device (as a described herein, e.g., such as a smart phone), wherein the computing device provides an interface to receive information provided by the patient/user, and also output information to the patient/user (e.g., via a display). The patient/user may be able to input specific patient information regarding the patient via the patient interface 108, such as gender, age, current symptoms, etc., wherein such information may be received by the AD App 106. In some cases, the patient/user can provide ECG data obtained from an external ECG device (e.g., from a third party ECG administrator, such as a doctor's office or other healthcare facility). In some cases, the ECG data is provided as a PDF document of the measured data, wherein the AD App is configured to extract the ECG data from the PDF document.

In some cases, the ECG data is obtained from a healthcare administrator via a healthcare administrator computing device 112, which may be in communication with the AD APP and other device (such as via a cloud server 114). The healthcare administrator may be a physician, a nurse, a physician's assistant, or other healthcare employee with access to the patient's information. In some cases, the health care administrator provides ECG data as a PDF file, which as described herein, the AD App may be configured to extract the measured data.

As described herein, the AD App 106 may use artificial intelligence, which may use a machine learning algorithm to determine a health characterization for a patient based on the ECG data and the patient information. In some cases, the AD App 106 will contain the machine learning algorithm and access trained data sets 110 via another computing device (e.g., via a cloud server 114) to evaluate the specific patient information received. As described herein, in some cases, the trained data sets comprises at least about 100, 1000, 10000, 20000, 500000 data sets. As described herein, each trained data set may comprise an ECG obtained from an individual. In some cases, each trained data set is correlated with a health characterization, such as a classification of a heart rhythm (e.g., sinus rhythm, atrial fibrillation).

In some cases, the machine learning algorithm is located with another computing device 100 comprising the trained data sets, wherein the AD App 106 relays the specific patient information (e.g., uncharacterized ECG data) to the machine learning algorithm 110 for evaluation. Thereby, using the AD App 106, the trained data sets and machine learning algorithm 110, the AD App 106 is able to characterize a patient health condition (such as heart rhythm), and output said characterization via the patient interface 108. In some cases, the characterization may be a classification of heart rhythm, such as an Atrial Fibrillation risk group, as described herein.

FIG. 2 provides another exemplary depiction of a system described herein, which is similar to FIG. 1 except that the ECG device 104 provides the ECG data to the AD App 106 instead of via another computing device (such as via cloud server 114). For example, the ECG device 104 may provide the ECG data (raw or processed) via the ECG App 102. In some cases, the ECG device 104 will directly provide 116 the ECG data to the AD App 106. In such cases, the ECG device 104 may be a part of a wearable device (e.g., smartwatch), wherein the wearable device also contains the AD App 106.

FIGS. 13A-15D provide an exemplary depiction of ECG data (e.g. ECG recording) over a 30 second interval that has been processed by the classifier. FIGS. 13A-13D depict an ECG data characterized as normal sinus rhythm. FIG. 13A depicts a graphical output of the ECG raw data, wherein the 30 second recording has been divided into six 5-second segments. The classifier may perform a first analysis of the (initially uncharacterized) ECG data based on the ECG raw data, using the trained data sets (as described herein), to provide a basic characterization of the heart rhythm (e.g., sinus rhythm, atrial fibrillation, etc.). FIG. 13B provides an exemplary depiction of a second analysis performed by the classifier, wherein in addition to the basic rhythm characterization of ECG data, the classifier further identifies secondary ECG characteristics as relating to analysis of P waves 1302, QRS complexes 1304, and T waves 1306. In some cases, the classifier may output corresponding intervals of the P waves (e.g., PR), T waves (e.g., QT), and QRS complexes (e.g., QRS width)—for example see FIG. 13D. The classifier may further identify QRS fragmentation (as described herein) based on the secondary ECG characteristics. FIG. 13C provides an exemplary depiction of a R-R interval graph which identifies the variability for the 30 second ECG recording. As described herein, the classifier may use summary statistics based on the R-R variability (via the R-R interval graph) to help characterize the heart rhythm (e.g., distinguish between sinus rhythm and atrial fibrillation). FIG. 13C further lists exemplary statistics of the RR interval (the mean R-R interval is 810 ms and the standard deviation is 12 ms (1.47% of 810 ms), as well as other patient information, such as sex, age, BMI, and any current symptoms.

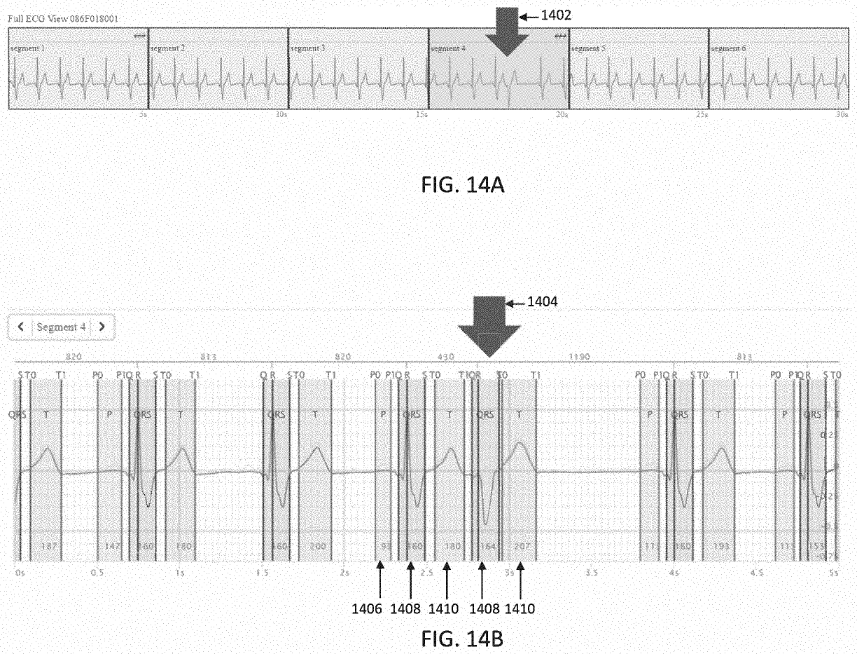

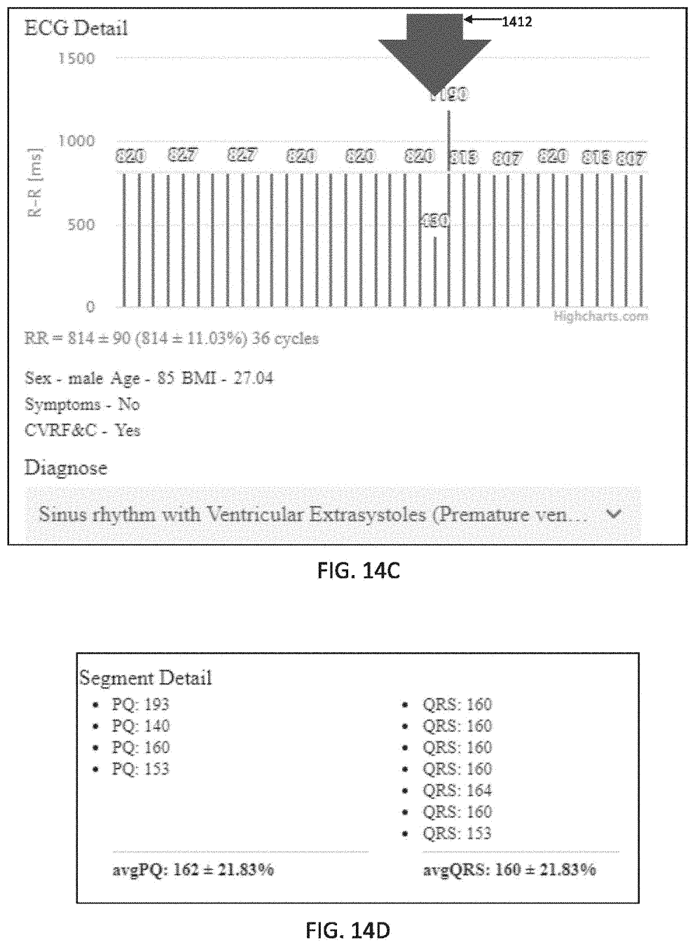

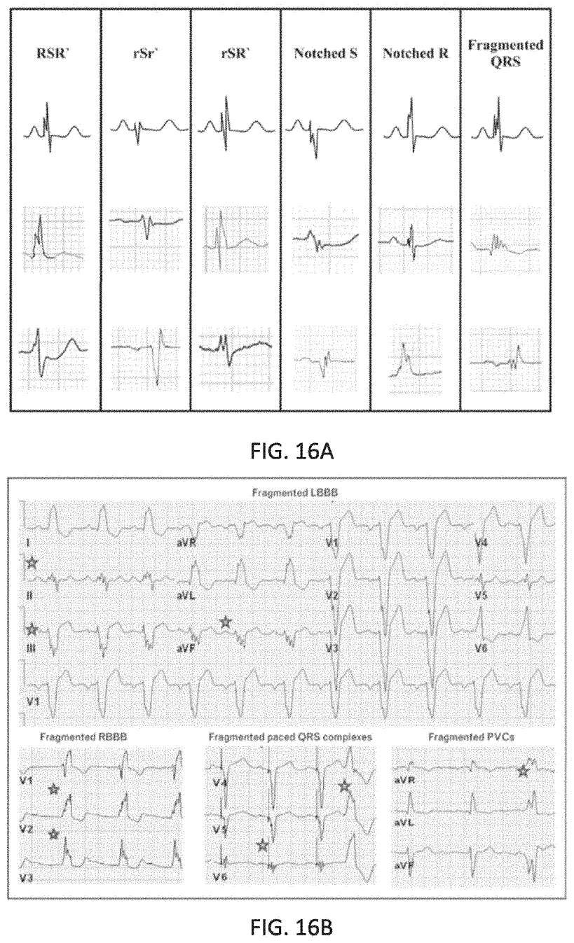

FIGS. 14A-14D similarly depict an ECG data characterized as sinus rhythm with ventricular extrasystoles (e.g., a ventricular extra beat among the sinus beats). FIG. 14A depicts a graphical output of the ECG raw data, wherein the ventricular extra beat is identified with arrow 1402. The classifier may perform a first analysis of the (initially uncharacterized) ECG data based on the ECG raw data, using the trained data sets (as described herein), to provide a basic characterization of the heart rhythm (e.g., sinus rhythm, atrial fibrillation, etc.). FIG. 14B provides an exemplary depiction of a second analysis performed by the classifier, wherein in addition to the basic rhythm characterization of ECG data, the classifier further identifies secondary ECG characteristics as relating to analysis of P waves 1406, QRS complexes 1408, and T waves 1410. In some cases, the classifier may output corresponding intervals of the P waves (e.g., PR), T waves (e.g., QT), and QRS complexes (e.g., QRS width)—for example see FIG. 14D. The ventricular extra beat is identified with arrow 1404. The classifier may further identify QRS fragmentation (as described herein) based on the secondary ECG characteristics. FIG. 16A provides examples of narrow-QRS fragmentation (depiction of RSR′ pattern and its variants) (reference: Das M K et al. Heart Rhythm 2007; 4:1385-92). FIG. 16B provides examples of Wide-QRS Fragmentation (reference: Das M K, El Masry H. Curr Opin Cardiol 2010; 25:59-64). FIG. 14C provides an exemplary depiction of a R-R interval graph which identifies the variability for the 30 second ECG recording. The ventricular extra beat is identified with arrow 1412. As depicted in FIG. 14C, a short-long pair of R-R intervals is caused by the extra beat (short RR) that is followed by a “compensatory pause” (long R-R) preceding the next sinus beat. As described herein, the classifier may use summary statistics based on the R-R variability (via the R-R interval graph) to help characterize the heart rhythm (e.g., distinguish between sinus rhythm and atrial fibrillation). FIG. 14C further lists exemplary statistics of the R-R interval (the mean R-R interval is 814 ms and the standard deviation is 90 ms (11.03% of 814 ms), as well as other patient information, such as sex, age, BMI, and any current symptoms.

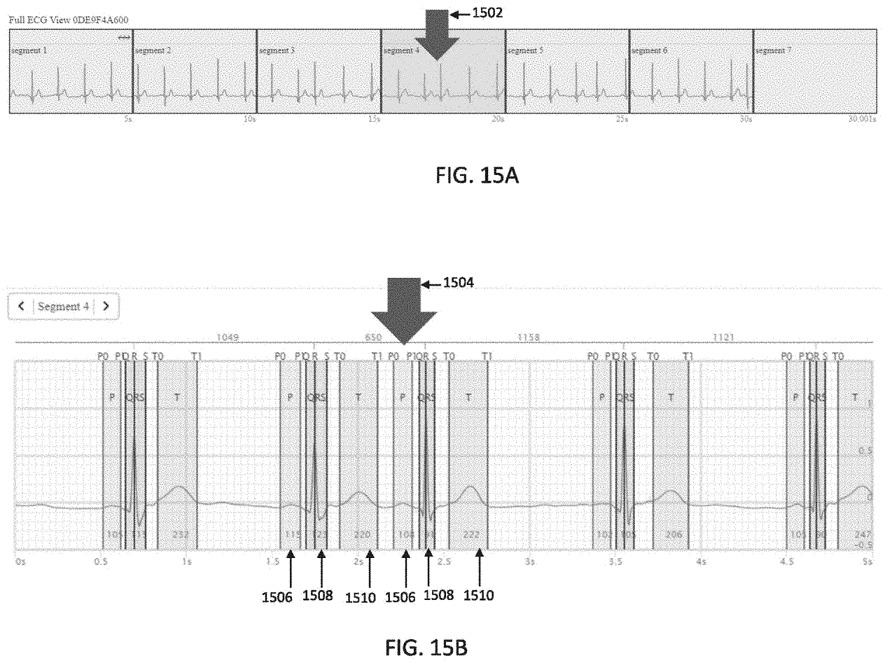

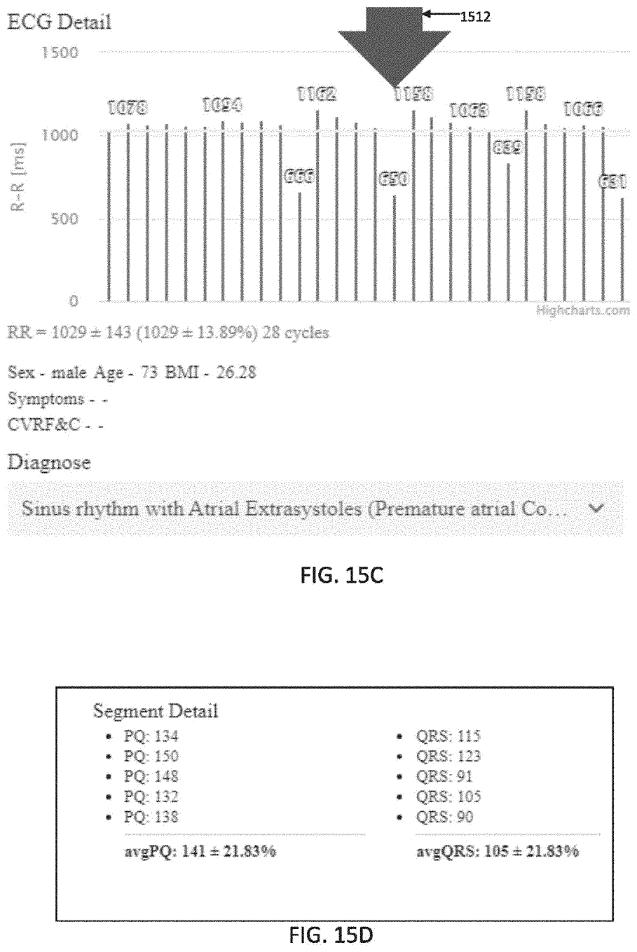

FIGS. 15A-15D similarly depict an ECG data characterized as sinus rhythm with atrial extrasystoles (e.g., an atrial extra beat among the sinus beats). FIG. 15A depicts a graphical output of the ECG raw data, wherein the ventricular extra beat is identified with arrow 1502. The classifier may perform a first analysis of the (initially uncharacterized) ECG data based on the ECG raw data, using the trained data sets (as described herein), to provide a basic characterization of the heart rhythm (e.g., sinus rhythm, atrial fibrillation, etc.). FIG. 15B provides an exemplary depiction of a second analysis performed by the classifier, wherein in addition to the basic rhythm characterization of ECG data, the classifier further identifies secondary ECG characteristics as relating to analysis of P waves 1506, QRS complexes 1508, and T waves 1510. In some cases, the classifier may output corresponding intervals of the P waves (e.g., PR), T waves (e.g., QT), and QRS complexes (e.g., QRS width)—for example see FIG. 15D. The ventricular extra beat is identified with arrow 1504. The classifier may further identify QRS fragmentation (as described herein) based on the secondary ECG characteristics. FIG. 15C provides an exemplary depiction of a R-R interval graph which identifies the variability for the 30 second ECG recording. The ventricular extra beat is identified with arrow 1512. As depicted in FIG. 14C, a short-long pair of R-R intervals is caused by the extra beat (short RR) followed by a “compensatory pause” (long R-R) preceding the next sinus beat. As described herein, the classifier may use summary statistics based on the R-R variability (via the R-R interval graph) to help characterize the heart rhythm (e.g., distinguish between sinus rhythm and atrial fibrillation). FIG. 15C further lists exemplary statistics of the R-R interval (the mean R-R interval is 1029 ms and the standard deviation is 143 ms (13.89% of 1029 ms), as well as other patient information, such as sex, age, BMI, and any current symptoms.

As described herein, the classifier (for example via the machine learning algorithm) may specify detection criteria for a given heart rhythm, which may correspond to a range of ECG data and parameters. The machine learning algorithm may adjust the detection criteria for a given hearth rhythm based on an ECG data that has been correctly assigned.

In some cases, all R-R intervals during sinus rhythm may need to be measured and the mean R-R interval as well as the standard deviation may be calculated for all 10-beats per minute (bpm) heart rate intervals according to Table 1 (R-R [in ms]=60,000/heart rate [in bpm]):

TABLE 1
R-R intervals Based on 10-Beats Per minute Heart Rate Intervals

| | Heart rate interval [bpm] | | | | | |
|---|---|---|---|---|---|---|
| | 30- | 40- | 50- | 60- | 70- | 80- |
| | 39.97 | 49.96 | 59.94 | 69.93 | 79.89 | 89.82 |
| R-R range [ms] | 1501- | 1201- | 1001- | 858- | 751- | 668- |
| | 2000 | 1500 | 1200 | 1000 | 857 | 750 |

| | Heart rate interval [bpm] | | | | | |
|---|---|---|---|---|---|---|
| | 90- | 100- | 110- | 120- | 130- | 140- |
| | 99.83 | 109.89 | 119.76 | 129.59 | 139.53 | 149.63 |
| R-R range [ms] | 601- | 546- | 501- | 463- | 430- | 401- |
| | 667 | 600 | 545 | 500 | 462 | 429 |

| | Heart rate interval [bpm] | | | | |
|---|---|---|---|---|---|
| | 150- | 160- | 170- | 180- | 190- |
| | 159.57 | 169.49 | 179.64 | 189.27 | 200 |
| R-R range [ms] | 376- | 354- | 334- | 317- | 300- |
| | 400 | 375 | 353 | 333 | 316 |

FIGS. 3A to 7B depict exemplary ECG data and corresponding analyses. Specifically, FIGS. 3A, 4A, 5A, 6A, and 7A depict ECG data corresponding to a type of heartbeat, while FIGS. 3B, 4B, 5B, 6B, and 7B depict an analysis from the corresponding ECG data. The ECG data for FIGS. 3A, 4A, 5A, 6A, and 7A are based on 30-second 1-channel electrocardiograms. FIGS. 3A, 4A, and 5A further list the R-R interval (as described herein). FIG. 3A also indicates the PQ and QT intervals. The ECG data may be obtained via the ECG app. The ECG data may be extracted by the AD app, and may further be outputted by the AD app (for e.g., FIGS. 3A, 4A, 5A, 6A, and 7A). The AD app may perform an analysis for the ECG data and output the analyses shown in FIGS. 3B, 4B, 5B, 6B, and 7B. The analysis of the ECG data may be performed by another computing device that receives the ECG data. As depicted, the analyses in FIGS. 3B, 4B, 5B, 6B, and 7B show column charts of R-R intervals (beat-to-beat intervals) measured in milliseconds (ms) from the corresponding ECG data.

The distribution of R-R intervals in an ECG data may be described by summary statistics, namely, by 1) the (arithmetic) mean and standard deviation (“SD”), 2) the median (also called the 50th percentile, since it is the R-R interval representing the half-way value of all R-R intervals ordered by size from smallest to largest) and 3) the 25th and 75th percentiles (also called 1st and 3rd quartiles). For example, in the ECG data in FIG. 3A, the mean R-R interval is 909 ms (corresponding to a mean heart rate of 66 beats per minute [bpm]) and the SD is 95 ms (10.4% of 909 ms). Another variable that may describe the distribution of R-R intervals is the root mean square of successive differences (“RMSSD”). The RMSSD may be calculated as the square root of the mean of all squared differences between successive R-R intervals, i.e., √(mean[ΔRR₁², ΔRR₂², . . . , ΔRRₙ₋₁²]), with ΔRRᵢ=R−Rᵢ−R−Rᵢ₊₁.

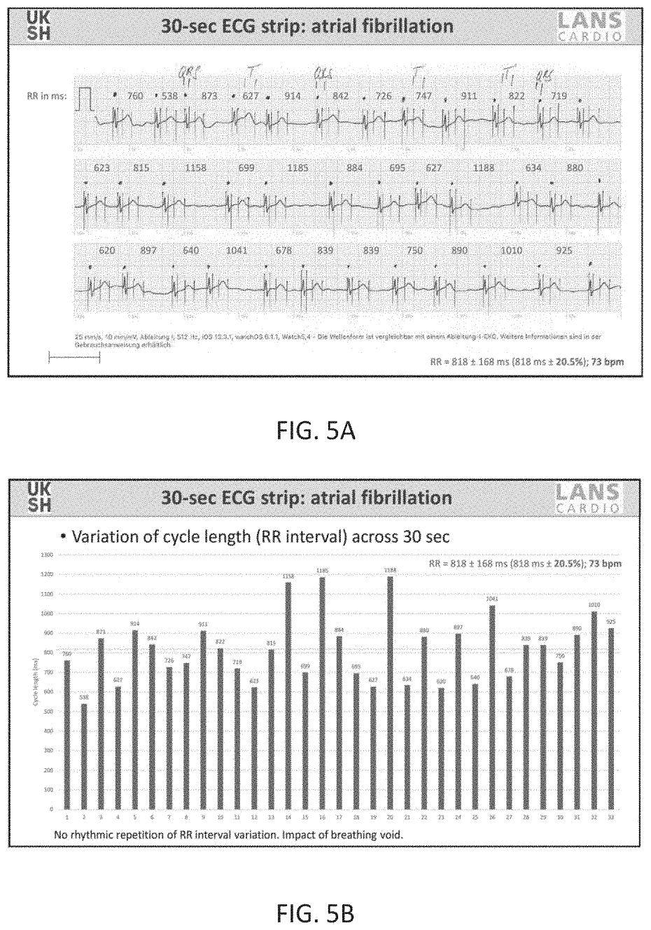

The distribution of R-R intervals may also be assessed by visualizing the curve enveloping the R-R interval columns. For example, as depicted in FIGS. 3B and 4B, the resulting column chart may approximate a sinusoidal curve in patients with a normal, regular heart rhythm (e.g., sinus rhythm [“SR”]) in which inspiration will shorten the R-R interval and expiration will prolong it. In contrast, as shown in FIG. 5B, this periodic variation of R-R intervals may disappear in patients with atrial fibrillation (“AF”), a heart rhythm disorder characterized by a highly irregular, and often rapid, heartbeat.

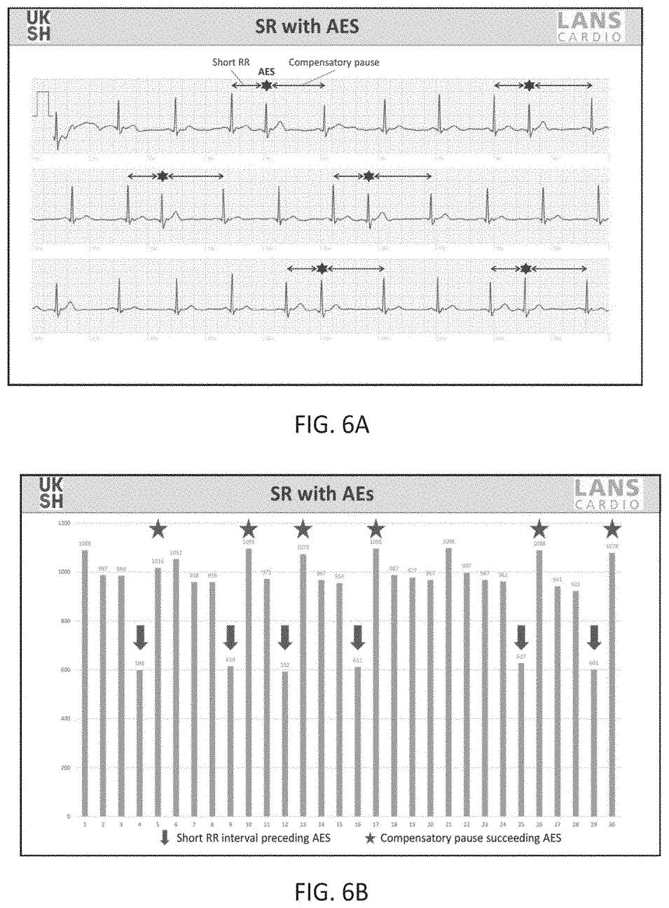

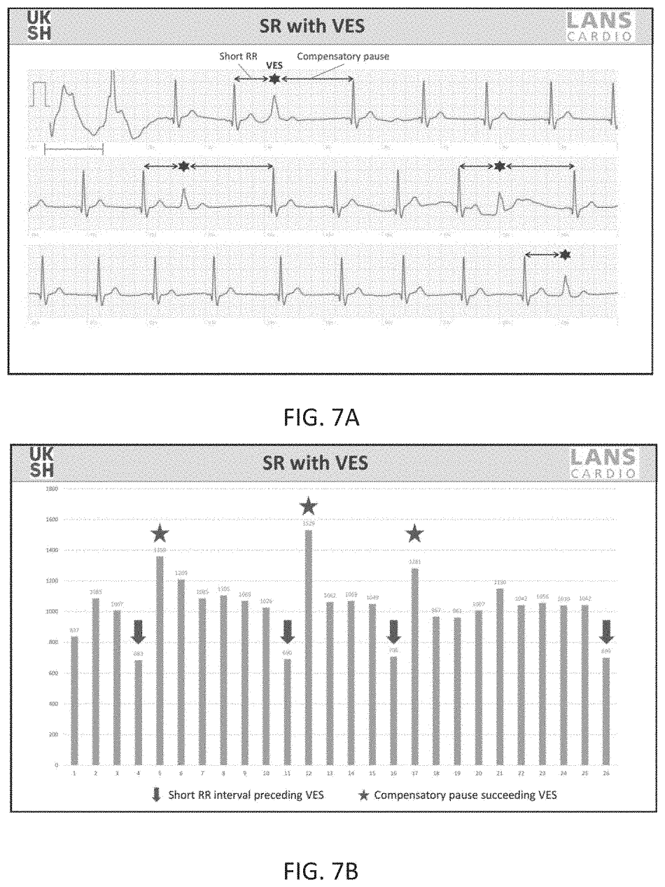

In patients predominantly in SR who experience (atrial or ventricular) premature beats (extrasystoles) at irregular intervals, a striking feature may be visible on their ECGs (ECG data) and R-R column charts. Each premature beat, be it atrial (e.g., FIG. 6A-B) or ventricular (e.g., FIG. 7A-B), is preceded by a short R-R interval of an almost fixed duration and succeeded by a “compensatory” extra-long R-R interval. The distinction between atrial and ventricular extrasystoles may be made by the duration of the QRS complex: if the QRS complex lasts 110 ms or less (“narrow” QRS complex), the extrasystole may originate from the atrium (atrial extrasystole [AES]); if the QRS complex lasts for more than 110 ms (“wide” QRS complex), the extrasystole may originate from the ventricle (ventricular extrasystole [VES]).

Described herein, is an exemplary process for differentiating atrial fibrillation (AF) from sinus rhythm (SR) via R-R interval parameters. First, ECG data is received, wherein all R-R intervals free from artifacts (e.g., free from disruptions such as noise, motion, or poor contact that affects the measured electrical activity of the heart) across 30 sec are measured (in milliseconds [ms]). The AD app as described herein may receive the ECG data. Next, the measured R-R intervals may be displayed as a column chart. Next, a summary description of each 30-second set of R-R intervals (for patients in SR as well as patients in AF) by mean and standard deviation (SD), median, 25th percentile, 75th percentile and RMSSD, may be calculated from all R-R intervals in the set. The outputting of the column chart of R-R intervals, and calculation of the statistics for the 30-second set of R-R intervals may be performed by the AD app or other computing device. To eventually differentiate AF from SR, “reference pools” (e.g., trained data sets) of sinus R-R intervals from at least about 100 or 500 SR electrocardiograms may need to be constructed. The reference pools may correspond to 30-second sets of ECG data for sinus rhythm. The reference pools of SR electrocardiograms may be verified by an experienced electrophysiologist. These reference pools may be stratified according to the mean heart rate (“HR”), e.g., by mean heart rate ranges (40 to less than 50 bpm, 50 to less than 60 bpm etc.). For each reference pool, the mean and SD, the median, the 5th and 95th percentile, as well as the mean[RMSSD] and SD[RMSSD] may be calculated for all R-R intervals in the pool. Each reference pool may comprise at least 50, 100, 200, 500, or 1000 SR electrocardiograms.

As such, for an unknown electrocardiogram (e.g., uncharacterized ECG) to be diagnosed (e.g., characterized), the mean, median and RMSSD of its R-R intervals may be compared with the HR-corresponding reference pool (e.g., reference pool with a mean HR corresponding to unknown electrocardiogram mean HR) as follows: 1) “Mean criterion”, wherein if the mean of the R-R intervals of the unknown ECG is within the HR-corresponding reference pool range defined by ‘reference mean±2× reference SD’, there is a 95% probability that the underlying rhythm is SR. If the mean exceeds the HR-corresponding reference pool range defined by ‘reference mean+2× reference SD’, then there is a 95% probability that the underlying rhythm is AF; 2) “Median criterion”, wherein if the median of the R-R intervals of the unknown ECG is inside the HR-corresponding reference pool range delimited by its 5th and 95th percentiles, there is a 95% probability that the underlying rhythm is SR. If the median exceeds the 95th percentile of the HR-corresponding reference pool range, then there is a 95% probability that the underlying rhythm is AF; and/or 3) “RMSSD criterion”, wherein if the RMSSD of the R-R intervals of the unknown ECG is within the HR-corresponding reference pool range defined by ‘reference mean[RMSSD]±2× reference SD[RMSSD]’, there is a 95% probability that the underlying rhythm is SR. If the RMSSD exceeds the HR-corresponding reference pool range defined by ‘reference mean[RMSSD]+2× reference SD[RMSSD]’, then there is a 95% probability that the underlying rhythm is AF.

The Mean criterion, Median criterion, RMSSD criterion, or any combination thereof, may subsequently be assessed in terms of sensitivity, specificity, and (positive and negative) predictive value using an electrophysiologist's characterization of the “unknown” ECG as a reference.

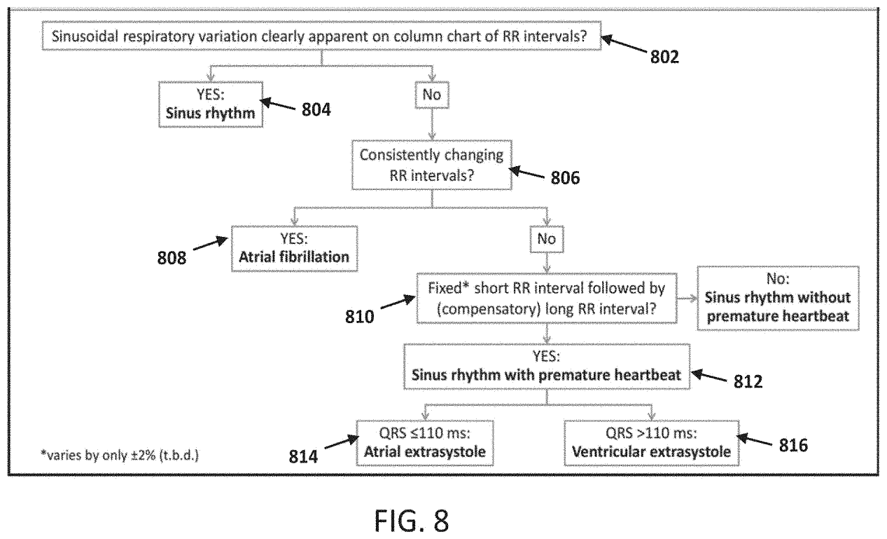

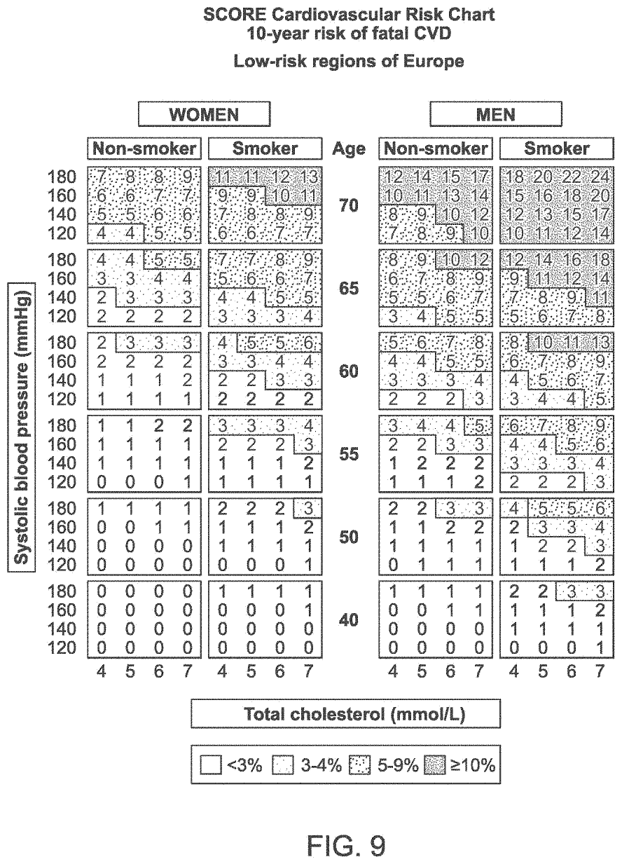

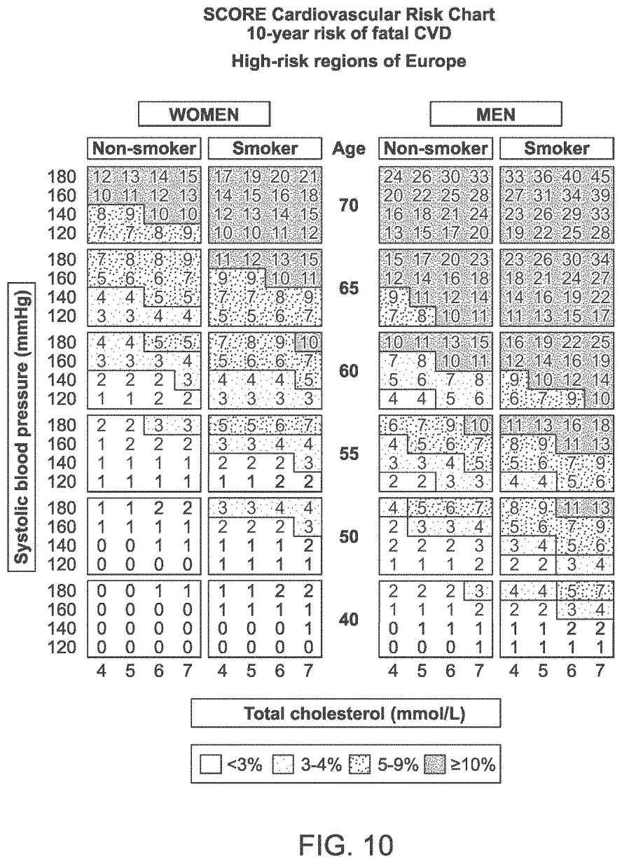

Another exemplary process for characterizing a heartbeat based on unknown ECG data is depicted in FIG. 8, wherein the process is based primarily on the unknown ECG data and corresponding R-R interval column chart (as described herein). The process may not require the summary statistics as described herein. The process may differentiate sinus rhythm from atrial fibrillation upon receiving ECG data, and may also categorize ECG data as corresponding to Atrial Extrasystole or Ventricular Extrasystole. The process may be at least partly performed by the AD app when obtaining ECG data, as described herein. For example, the AD app or other computing device may first process the ECG data to create a corresponding R-R interval column chart, as described herein. The column chart may then be used by the AD app, other computing device, or a user, (e.g., physician, medical staff) to evaluate and/or classify the heartbeat. For example, the R-R column chart may be used to determine whether a sinusoidal respiratory variation is clearly apparent (e.g., a sinusoidal curve) 802. If a sinusoidal curve is apparent, the unknown ECG data corresponds to Sinus rhythm 804. If a sinusoidal curve is not apparent, the R-R column chart may be used to determine whether consistently changing R-R intervals are depicted (e.g., irregular R-R intervals) 806. If irregular R-R intervals are depicted, the unknown ECG data corresponds to Atrial fibrillation 808. If consistently changing R-R intervals are not depicted in the column chart, then the R-R column chart may be used to determine whether the unknown ECG data corresponds to Sinus rhythm with a premature heartbeat 812, if a fixed short R-R interval is followed by a long (compensatory) R-R interval 810. A short R-R interval may vary (for example, for several premature heartbeats) by only about +/−2%, 5%, or 10%. If a Sinus rhythm with premature heartbeat is identified, the unknown ECG may be identified as originating from the atrium or the ventricle by measuring the duration/width of the QRS interval of the extrasystole (QRS may be from the ECG data). The extrasystole may be identified as corresponding to 1) Atrial extrasystole (i.e., originating from the atrium) if the QRS interval of the extrasystole is less than or equal to 110 ms 814, or 2) Ventricular extrasystole (i.e. originating from the ventricle) if the QRS interval of the extrasystole is greater than 110 ms 816. In some instances, if a P wave precedes the premature QRS complex, this may also correspond to an atrial extrasystole. As aforementioned, this process may be performed at least partly by the AD app, any computing device, or a user.

All other user information required for the classifier analysis may be collected and processed via the AD app and corresponding cloud environment (i.e., network).

Personal data of the user may be saved directly on the computing device comprising the AD app, such as a smartphone, tablet, computer, etc., and may be configured to be protected from data loss, theft and manipulation, e.g. by comparing the IP address. All personal data may be pseudonymized before getting transmitted to the cloud network. If the user changes his/her personal information or the current ECG shows major differences to the one before, a consistency check may be carried out. A consistency check may be performed based on the amplitude and morphology of the QRS complex. All communication may be secured by using end-to-end encryption. The server infrastructure may be globally available, fail-safe due to redundancy, and scalable to address all future data needs of studies and users. Data on the server may be stored securely with technologies such as block-chain technology. Data may be stored in a structured way to be analyzed by the classifier as well as by a cardiologist. The ECG data may be digitized and the classifier may systematically diagnose (e.g., characterize) said ECG data according to a workflow.

The AD app may be synchronized with the ECG app immediately when an ECG is recorded, such that the AD app may receive the ECG data immediately when it is recorded.

Users may be able to send ECGs to a recipient from anywhere in the world through an internet connection. If the user cannot send the raw ECG data immediately to the cloud network or a network error occurs, the raw ECG data may be saved on the computing device comprising the AD app (e.g., smartphone, tablet, smart watch, computer, or combinations thereof) and the AD app may try to send the ECG data when the computing device detects an internet connection, to prevent loss of the current ECG data.

A cardiologist may be required to approve the classifier characterization. The technical infrastructure may be capable of and open to different workflows, for instance by enabling or disabling an approval function by a cardiologist. Feedback for the classifier characterization, such as from a cardiologist, may be received immediately upon entry of such feedback. If a cardiologist needs to be involved in a workflow, a worldwide allocation system may be implemented where a cardiologist is in charge of reviewing the user data. All transmission and response times are tracked for evaluation. Cardiologists may authorize or correct the characterization of the classifier. To change a characterization, the cardiologist may choose from a certain number of pre-defined diagnoses.

### Machine Learning Algorithm

As described herein, the classifier may use a machine learning algorithm to the data sets of patient information (as described herein) for characterizing a patient (e.g., characterizing a patient's heart rhythm). As described herein, the patient information may include any combination of ECG data, gender, age, current symptoms, etc. In some cases, the machine learning algorithm may need to extract and draw relationships between patient information (such as ECG data) as conventional statistical techniques may not be sufficient. In some cases, machine learning algorithms may be used in conjunction with conventional statistical techniques. In some cases, conventional statistical techniques may provide the machine learning algorithm with preprocessed data sets.

In some cases, as described herein, the plurality of data sets (of patient information) may be classified into any number of categories. For example, the categories may be classified as normal sinus rhythm as well as three cardiac arrhythmias: atrial fibrillation, atrial extrasystoles and ventricular extrasystoles, etc.

In some cases, select patient information may be discarded prior/during machine learning classification. The select patient information being discarded may be by a person, such as a physician, or may be by a computing device (e.g., via the AD app). In some cases, the select patient information may be discarded based on a threshold value, so as to help remove outliers or inaccurate information from skewing the trained data sets.

In some cases, the machine learning algorithm may be, for example, an unsupervised learning algorithm, supervised learning algorithm, or a combination thereof. The unsupervised learning algorithm may be, for example, clustering, hierarchical clustering, k-means, mixture models, DBSCAN, OPTICS algorithm, anomaly detection, local outlier factor, neural networks, autoencoders, deep belief nets, Hebbian learning, generative adversarial networks, self-organizing map, expectation-maximization algorithm (EM), method of moments, blind signal separation techniques, principal component analysis, independent component analysis, non-negative matrix factorization, singular value decomposition, or a combination thereof. The supervised learning algorithm may be, for example, support vector machines, linear regression, logistic regression, linear discriminant analysis, decision trees, k-nearest neighbor algorithm, neural networks, similarity learning, or a combination thereof. In some embodiments, the machine learning algorithm may comprise a deep neural network (DNN). The deep neural network may comprise a convolutional neural network (CNN). The CNN may be, for example, U-Net, ImageNet, LeNet-5, AlexNet, ZFNet, GoogleNet, VGGNet, ResNet18 or ResNet, etc. Other neural networks may be, for example, deep feed forward neural network, recurrent neural network, LSTM (Long Short Term Memory), GRU (Gated Recurrent Unit), Auto Encoder, variational autoencoder, adversarial autoencoder, denoising auto encoder, sparse auto encoder, Boltzmann machine, RBM (Restricted BM), deep belief network, generative adversarial network (GAN), deep residual network, capsule network, or attention/transformer networks, etc.

In some cases, the machine learning algorithm may be, for example, a Naïve Bayes classifier, linear regression, logistic regression, decision trees, random forests, rotation forests, K nearest neighbors (KNN), clustering, support vector machines (SVM), or neural networks. In some cases, the machine learning algorithm may include ensembling algorithms such as bagging, boosting, and stacking. The machine learning algorithm may be individually applied to the plurality of data sets (including for example each ECG data extracted for a given person/patient), such that each data set may have a separate iteration of the machine learning algorithm, or all the plurality of data sets may be applied at once.

In some cases, as described herein, the ECG data signals may be collected over a single channel ECG or a multichannel ECG having a plurality of channels. The machine learning algorithm may be individually applied to the ECG data extracted for each channel, such that each channel has a separate iteration of the machine learning algorithm, or the machine learning algorithm is applied to the plurality of ECG data extracted from all channels or a subset of channels at once.

In some embodiments, the machine learning algorithm may have a variety of parameters. The variety of parameters may be, for example, learning rate, minibatch size, number of epochs to train for, momentum, learning weight decay, or neural network layers etc.

In some embodiments, the learning rate may be between about 0.00001 to 0.1.

In some embodiments, the minibatch size may be at between about 16 to 128.

In some embodiments, the neural network may comprise neural network layers. The neural network may have at least about 2 to 1000 or more neural network layers.

In some embodiments, the number of epochs to train for may be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 500, 1000, 10000, or more.

In some embodiments, the momentum may be at least about 0.1, 0.2, 0.3, 0.4, 0.5, 0.6, 0.7, 0.8, 0.9 or more. In some embodiments, the momentum may be at most about 0.9, 0.8, 0.7, 0.6, 0.5, 0.4, 0.3, 0.2, 0.1, or less.

In some embodiments, learning weight decay may be at least about 0.00001, 0.0001, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, or more. In some embodiments, the learning weight decay may be at most about 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.0001, 0.00001, or less.

In some embodiments, the machine learning algorithm may use a loss function. The loss function may be, for example, regression losses, mean absolute error, mean bias error, hinge loss, Adam optimizer and/or cross entropy.

In some embodiments, the parameters of the machine learning algorithm may be adjusted with the aid of a person/user and/or computer system (e.g., a computing device as described herein).

In some embodiments, the machine learning algorithm may prioritize certain characteristics (see FIGS. 13-15 for example) of the trained data sets (e.g., ECG recordings). The machine learning algorithm may prioritize trained data sets having characteristics (e.g., QRS complexes, R-R intervals) that may be more relevant for detecting one or more hearth rhythms associated with determining a medical condition (for example, stroke risk, cardiovascular age, etc.). The trained data set may be more relevant for detecting one or more cardiovascular conditions if the trained data set (e.g., particular ECG data) is classified more often than another type of ECG recording (or another ECG data characteristic). In some cases, the trained data set may be prioritized using a weighting system. In some cases, the trained data set may be prioritized on probability statistics based on the frequency and/or quantity of occurrence of a characteristic of the ECG data set. The machine learning algorithm may prioritize trained data sets with the aid of a human and/or computer system.

In some cases, the machine learning algorithm or conventional statistical techniques are configured to determine if a trained data set contains outliers, so as to reject such particular outliers from being considered when being evaluated by the machine learning algorithm. In some cases, the machine learning algorithm may prioritize certain trained data sets and/or patient information to reduce calculation costs, save processing power, save processing time, increase reliability, or decrease random access memory usage, etc.

### AD App

The AD app may comprise of three main parts displayed in a menu bar at the bottom of the Main Page of the AD app, as provided on a corresponding display of a computing device (e.g., smartphone, tablet, wearable computing device, computer): “ECG”, “Personal Health Risks” and “Settings”.

The main page of the AD App may display a preview of the latest ECG as well as the latest recommendation related to it, the cardiovascular risk age, risk of stroke, risk of sudden cardiac death and the ECG status bar. Stroke risk and risk of sudden cardiac death may be given in percentages, while a cardiovascular risk age may be provided as a number, as provided in tables herein. The ECG status bar may show the time until the next ECG needs to be created depending on the user's Atrial Fibrillation (AF) risk group (see Tables 5-6 herein). The ECG menu status bar on the main page may change filling status and color from green to yellow to red, wherein the preview of the ECG archive may highlight earlier ECGs in red (emergency situation), yellow (cardiac arrhythmia but no emergency), green (sinus rhythm) based on the characterization.

In the ECG menu page, all reports may be archived including ECG diagnoses (e.g., characterizations) and recommended actions. The reports may have a date and time stamp and are listed according to the day of recording. By selecting the calendar icon at the top of a display of the AD app, a specific ECG may be searched for by entering a specific day or by clicking on a calendar date. Days where an ECG was made are highlighted in the calendar. By selecting a single report, the ECG tracing, the personal health risks at the time of creation and the clinical recommendation may be seen. The ECG can be expanded to full screen and all parts of the ECG may be viewed in detail.

The Personal Health Risks menu page may feature the cardiovascular risk age of the user, the risks of stroke and sudden cardiac death of the user, as well the associated recommendations. The corresponding numbers may be shown after the evaluation phase of 4 weeks is over. Before this time, a loading bar may visualize the time until the end of the evaluation phase. In the personal health risk menu page, the risk factors may be displayed in a diagram. The classification of the user, based on all risk assessments, may be highlighted.

The Settings menu page may include the user's profile, the subscription status, a user's guide, language settings as well as all legally required documents and information. In the user's profile, the user may be asked to provide his medical history including the current medication. Medical history may comprise the collected non-personal data described herein, such as biometric data and/or medical data, which may comprise height, weight, waist circumference, smoking status, country of residence, total cholesterol, LDL cholesterol, systolic blood pressure, medication, congestive heart failure, ischemic cardiomyopathy, nonischemic cardiomyopathy, NYHA functional class, LV ejection fraction, hypertension, diabetes mellitus, glycated hemoglobin (HbA1c), vascular disease, prior TIA/stroke, prior myocardial infarction, valvular heart disease, pacemaker, syncope, or combinations thereof. Current medication may be imported from an external directory. Some fields are mandatory for getting an ECG characterization, some are only relevant for the calculation of the personal health risks.

Users should create an ECG when recommended by the AD app (basis is their AF risk group as provided in Tables 5-6 herein) or when they have symptoms, especially as described herein, which comprise chest pain, palpitations, rapid heart rate, dyspnea, dizziness, presyncope, syncope, sweating, speech disorder, weakness/paralysis, or combinations thereof. When sending the ECG data to the cloud network, the user may be asked to report his/her symptoms. If the ECG cannot be evaluated by the classifier, the user may be asked to retry (up to three times). When no characterization is possible three times in a row, the user may be advised to contact designated personnel that may provide assistance.

The estimated cumulative risk (in %) of fatal cardiovascular disease within 10 years may be determined by the (Systematic Coronary Risk Estimation (SCORE) system (reference: 2019 ESC/EAS Guidelines for the management of dyslipidaemias. Eur Heart J 2020; 41:111-88). The SCORE risk of cardiovascular death may be based on the following factors: country of residence (there are countries at low and high cardiovascular risk), age, sex, smoking status, systolic blood pressure, and total cholesterol. As depicted in FIGS. 1-2, exemplary SCORE charts are provided for low risk populations, and high risk populations (reference: 2019 ESC/EAS Guidelines for the management of dyslipidaemias. Eur Heart J 2020; 41:111-88). The chart allows to estimate the relative risk of the user, meaning that a person in the top right corner (e.g., a 70-year old male smoker from a low-risk country with a systolic BP of 180 mmHg and a total cholesterol of 7 mmol/l (270 mg/dl)) is at a 24 times higher risk of cardiovascular death than a person with a 1% risk (e.g., a 50-year old male non-smoker from a low-risk country with a slightly increased total cholesterol as the only risk factor).

Instead of the SCORE risk, a cardiovascular risk age (CVRA) may be used in the AD app because it is an intuitive and easily understood variable for communicating the user's cardiovascular risk, especially to younger people. A young person with many risk factors may have the same risk as an older person with no risk factors. The AD app may correlate a given SCORE risk with a corresponding CVRA, and may further adjust the CVRA based on activities by a patient (such as reducing smoking, increasing exercise, etc). The assignment of the CVRA has been simplified and generalized such that it can be specified regardless of gender and country of residence. Rather than the highest possible age of a specific SCORE risk, an average value is used to “reward” and motivate older users for their healthy lifestyle with a lower CVRA than their actual age. Based on the calculated SCORE risk, the AD app may be able to display the cardiovascular risk age (CVRA), as shown in Table 2. Users under the age of 40 may not get a CVRA but the app may tell them that they are at a “low risk” of cardiovascular death within the next 10 years. All users over 70 years may be classified as “high risk” and may not be assigned a CVRA either.

TABLE 2
CVRA Calculated Based on SCORE Risk

| SCORE risk (%) | CVRA (years)* |
|---|---|
| 0 | 40 |
| 1 | 45 |
| 2 | 50 |
| 3 | 55 |
| 4 | 60 |
| 5 | 65 |
| 6, . . . , 9 | 70 |
| ≥10 | high risk |

The AD app may provide recommendations based on the SCORE risk and untreated blood concentration of low-density lipoprotein (LDL) cholesterol according to Table 3 (reference: Kaplan R M et al. Circulation. 2019; 140:1639-46).

TABLE 3
Lifestyle Change Recommendation based on SCORE and LDL-C

| | SCORE risk | LDL-C | |
|---|---|---|---|
| | (%) | (mg/dl) | Recommendation |
| Primary prevention: | | | Lifestyle changes |
| | <1 | 55 < 116 | Cardiologist consultation |
| | ≥1 < 5 | 55 < 100 | not necessary |
| | ≥5 < 10 | 55 < 70 | |
| | ≥10 | <55 | |
| Primary prevention: | | | Lifestyle changes |
| | <1 | <190 | Should consult cardiologist |
| | ≥1 < 5 | 100 < 190 | |
| | ≥5 < 10 | 70 < 100 | |
| | ≥10 | 55 < 70 | |
| Secondary prevention: | | | |
| | | <55 | |
| Primary prevention: | | | Lifestyle changes |
| | <1 | ≥190 | Must consult cardiologist |
| | ≥1 < 5 | ≥190 | |
| | ≥5 < 10 | ≥100 | |
| | ≥10 | ≥70 | |

| Secondary prevention: | |
|---|---|
| | ≥55 |

The lifestyle advices may be based on the values to be improved in the SCORE. They may be pre-defined and may be regularly updated based on guideline-directed treatment targets and goals for the prevention of cardiovascular diseases. The current guidelines are listed in table 4 (reference Kaplan R M et al. Circulation. 2019; 140:1639-46).

TABLE 4
Guidelines for Preventing Cardiovascular Diseases

| | |
|---|---|
| Smoking | No exposure to tobacco in any form. |
| Diet | Healthy diet low in saturated fat with a focus on wholegrain |
| | products, vegetables, fruit, and fish. |
| Physical | 3.5-7 h moderately vigorous physical activity per week or |
| activity | 30-60 min most days. |
| Body | BMI 20-25 kg/m², and waist circumference <94 cm (men) |
| weight | and <80 cm (women). |
| Blood | <140/90 mmHg. |
| pressure | |
| Diabetes | HbA1c: <7% (<53 mmol/mol). |
| LDL-C | Very-high risk in primary or secondary prevention: A |
| | therapeutic regimen that achieves ≥50% LDL-C reduction |
| | from baseline and an LDL-C goal of <55 mg/dl. |
| | High risk: A therapeutic regimen that achieves ≥50% LDL-C |
| | reduction from baseline and an LDL-C goal of <70 mg/dl. |
| | Moderate risk: A goal of <100 mg/dl. |
| | Low risk: A goal of <116 mg/dl. |

### Stroke Risk Determination

The annual stroke risk may be determined by two factors: CHA₂DS₂-VASc score and AF risk group. The CHA₂DS₂-VASc score represents the annual stroke risk in %. By giving all relevant information in the user's profile (Age, Gender, Congestive Heart Failure y/n, Hypertension y/n, Vascular disease y/n, Diabetes mellitus y/n, Stroke/TIA/Thromboembolism history y/n), the CHA₂DS₂-VASc score for the user can be calculated. In combination with the Atrial Fibrillation (AF) risk group, as defined in Table 6, the stroke risk of the user may be assessed according to Table 5, wherein SR represents sinus rhythm, and AF represents the Atrial Fibrillation risk group. (Reference: Kaplan R M et al. Circulation. 2019; 140:1639-46). According to the ROCKET-AF trial (Steinberg B A et al., Eur Heart J. 2015; 36:288-96), patients with persistent AF may carry a higher risk of stroke or systemic embolism (HR 1.27 [95% CI, 1.00-1.59]) than patients with paroxysmal AF. The stroke risk must be updated when one of the parameters change.

TABLE 5
Stroke Risk Estimation

| | | Risk group | | | |
|---|---|---|---|---|---|
| | | SR | AF I | AF II | AF III |
| CHA2DS2-VASc | 0 | 0.33% | 0.52% | 0.86% | n.a. |
| | 1 | 0.62% | 0.32% | 0.50% | n.a. |
| | 2 | 0.70% | 0.62% | 1.52% | n.a. |
| | 3 | 0.83% | 1.28% | 1.77% | n.a. |
| | 4 | 0.83% | 1.28% | 1.77% | n.a. |
| | ≥5 | 1.79% | 2.21% | 1.68% | n.a. |

Table 6 provides exemplary follow-up actions by a user based on AF risk group classification, which is based on 30-second ECGs (Reference: Modified from Kaplan R M et al. Circulation. 2019; 140:1639-46). In some cases, the AD app may determine the AF group classification according the trained data sets in evaluating the ECG data (via the classifier for example). After an initial ECG, an evaluation phase of 1 week may start. During this time, the user may need to send one ECG per day to the AD app, wherein the AD app may utilize the classifier to automatically analyze the ECG data. In case no irregularities are found (only sinus rhythm on the ECG), the user may be categorized in “SR group” and may need to send one ECG per month. If any of the ECGs in the evaluation phase or at any later time show AF, the user may be categorized in risk group “AF I” (paroxysmal AF [PAF] 6 min-23.5 h) and may need to send an ECG every 6-12 hours for the next 24 hours. Should they all still show AF, the user may move to risk group “AF II” (PAF>23.5 h) and may need to send an ECG every 24 hours for the next 7 days to check for persistent AF. If sinus rhythm is diagnosed at any time during the 7 days, the user remains in group “AF II” and the 7-day period starts new (to be repeated for a maximum of 4 weeks). Once all 7 ECGs show AF, the patient may be categorized in risk group “AF III” (persistent AF). After the 7 days, patients in group “AF III” should consistently send one ECG per week for a maximum of 3 months. Within these 3 months the medical decision may need to be made to either leave the patient in AF (permanent AF) or restore SR. If the patient is left in AF, he/she should send an ECG at least every 3 months. The user may move back to “SR group” if sinus rhythm is successfully restored by therapeutic intervention (e.g., cardioversion, medication, catheter ablation).

TABLE 6
Cardiac Rhythm Group Classification

| Rhythm | Group | Group classification based on 30-sec ECGs |
|---|---|---|
| Sinus rhythm | SR | First ECG shows SR → If asymptomatic: daily |
| | | ECG for 7 days or at any time of palpitations |
| PAF 6 min- | AF I | First ECG with AF, creation of 3 more ECGs |
| 23.5 h | | after 6, 12 and 24 h; if only the first and/or |
| | | second ECG show AF, patient remains in group |
| | | “AF I”; if all 3 ECGs show AF, patient moves |
| | | to category “AF II” |
| PAF >23.5 h | AF II | Daily ECG for 7 more days; if all ECGs show |
| | | AF, patients moves to category “AF III”. |
| Persistent | AF III | ECG 1x per week |
| AF >7 d | | |

Table 7 provides exemplary instructions the user may receive based on the ECG analysis (Stroke/Transient Ischemic Attack (TIA) prevention).

TABLE 7
Recommendations for Stroke/TIA prevention
based on ECG Analysis

| ECG diagnosis | | |
|---|---|---|
| (e.g. | | |
| characterization) | Acute symptoms | Recommendation |
| Sinus rhythm | No or mild symptoms such | Physician consultation |
| (with or without | as palpitations | not necessary |
| atrial extrasystoles | | |
| [AES]) | | |
| Atrial fibrillation | No or mild-to-moderate | Consult cardiologist |
| | symptoms such as | within maximally 24 |
| | palpitations/rapid | hours |
| | irregular heart rate, | |
| | dyspnea | |
| Atrial fibrillation | Major symptoms such as | Call ambulance or visit |
| | rapid irregular heart | emergency room of |
| | rate, dizziness, dyspnea/ | nearest hospital |
| | orthopnea, presyncope, | |
| | syncope, sweating | |

### Sudden Cardiac Death (SCD) Determination

The risk of sudden cardiac death may be dependent on selected subject/patient groups and certain age ranges, as described herein.

### Healthy Middle-Aged Subjects

An assessment of the 5- and 10-year risk of sudden cardiac death in the general population (n=6,830 subjects; mean age 51±14 years; 45.5% men; assessed at baseline in 1978-1980 [Mini-Finland Health Survey, MFHS]) can be based on the number of abnormalities the classifier detected in the latest ECG (reference: Holkeri A et al. Heart. 2020; 106:427-33). ECG abnormalities include 1) heart rate>80 bpm (mean RR<750 ms), 2) PR duration>220 ms, 3) QRS duration>110 ms, 4) left ventricular (LV) hypertrophy and 5) T-wave inversion. The abnormalities of ECG may be determined via the classifier, as described herein (for example, by using the trained data sets). The number of abnormalities will accumulate to the risk of SCD according to Table 8 below. In the MFHS, subjects with >3 ECG abnormalities had a hazard ratio for SCD of 10.23 (95% confidence interval, 2.29 to 19.8) compared with subjects without ECG abnormalities. The risk will be regularly updated.

Other ECG abnormalities may not be assessed from a single-channel ECG. For example, LV hypertrophy—which had a prevalence of 13.9% in the MFHS, second only to heart rate>80 bpm (prevalence 15.6%)—cannot be detected on a single-channel ECG. Therefore, healthy middle-aged app users in whom at least 1 of the other 4 ECG abnormalities was detected, need to receive an alert from the app to see a cardiologist shortly and have the presence of LV hypertrophy confirmed or excluded.

TABLE 8
SCD Risk for Healthy Middle-Aged Subjects in the General
Population Based on the Number of ECG Abnormalities

Risk of SCD

| | | 5-year risk | 10-year risk |
|---|---|---|---|
| Number of ECG | 0 | 0.3% | 0.7% |
| abnormalities | 1 | 1.7% | 3.3% |
| | 2 | 3.7% | 9.3% |
| | ≥3 | 13.1% | 25.8% |

Patients with Underlying Heart Disease

The risk of sudden cardiac death (SCD) was assessed in patients (92% men) with severely reduced Left ventricular ejection fraction (LVEF) and an implanted cardioverter-defibrillator (ICD) for primary or secondary prevention of SCD whose age ranged between 50 and 74 years. The risk was calculated from the number of appropriate ICD shocks as a surrogate of SCD; it therefore overestimates the actual risk of SCD. Given this limitation, the estimated risks of SCD according to the presence or absence of fractionated QRS complexes (fQRS) are given in Table 9 below for patients with coronary artery disease, and Table 10 below for patients with nonischemic cardiomyopathy (reference Das M K et al. Heart Rhythm 2010; 7:74-80), wherein mean LVEF was 26.8±10.4% in 84 patients with fQRS and 31.4±13.0% in 100 patients without fQRS.

TABLE 9
SCD Risk for Patients with Coronary Artery
Disease Based on ECG Abnormalities

Point Estimates of SCD

| | 6 M | 1 Yr | 18 M | 2 Yr |
|---|---|---|---|---|
| Non-fQRS (n = 69) | 2.9% | 4.7% | 4.7% | 12.1% |
| fQRS (n = 53) | 18.9% | 29.8% | 43.0% | 50.9% |

TABLE 10
SCD Risk for Patients with Nonischemic Cardiomyopathy
Based on ECG Abnormalities

Point Estimates of SCD

| | 6 M | 1 Yr | 18 M | 2 Yr |
|---|---|---|---|---|
| Non-fQRS (n = 31) | 0% | 0% | 7% | 7% |
| fQRS (n = 31) | 26.6% | 40.3% | 45.9% | 51.2% |

Patients with Ventricular Extrasystoles (VES)

If the user is a man between 35 and 57 years of age and has a single VES on his ECG, his risk of sudden cardiac death within 7.5 years may be 3 times higher than if he had no VES. In the presence of 2 or more VES, the risk may be about 4-fold higher (reference: Abdalla I S et al. Am J Cardiol 1987; 60:1036-42).

Patients with Non-Sustained Ventricular Tachycardia (VT)

In patients with ischemic and nonischemic cardiomyopathy and a reduced LVEF (ranging from 19% to 42%), the risk of SCD within 13 to 60 months may be increased by a factor of almost 3 if the ECG shows nonsustained VT (reference According to a meta-analysis by de Sousa M R et al. Eur J Heart Fail 2008; 10:1007-14).

### Recommendations for Prevention of SCD

The AD app may provide the recommendations listed in table 11 below for the prevention of sudden cardiac. ECG abnormalities are: 1) QRS>110 ms; QTc>440 ms (men); QTc>460 ms (women); 2) T-wave inversion; and 3) QRS fragmentation. Users may be followed clinically once per year to assess their health status and their true incidence of SCD. The users may be notified by the AD app to complete a health status questionnaire via the AD app.

TABLE 11
Recommendations for SCD Prevention Based on ECG Analysis

| ECG diagnosis | | |
|---|---|---|
| (e.g., | | |
| characterization) | Acute symptoms | Recommendation |
| Sinus rhythm | No or mild symptoms such | Physician consultation |
| | as palpitations | not necessary |
| At least 1 ECG | No or mild-to-moderate | Consult cardiologist to |
| abnormality and/ | symptoms such as | confirm/exclude |
| or VES and/or | palpitations/irregular | underlying heart disease |
| nonsustained | heart rate, dyspnea | |
| VT <180 bpm | | |
| Nonsustained | Major symptoms such as | Call ambulance or visit |
| VT ≥180 bpm or | rapid heart rate, | emergency room of |
| sustained VT | dizziness, dyspnea, | nearest hospital |
| (30 seconds) | presyncope, syncope, | |
| | sweating | |

### Additional AD App Features:

The AD app may be configured to provide push notifications that may be sent when: 1) ECG needs to be recorded (according to the AF risk group), 2) symptoms report needs to be sent, 3) user profile needs to be updated (no update in the last 4 weeks), 4) characterization is available, and/or 5) a personal health risk was updated. The AD app may also inform the user via push messages if a security-relevant update has been provided.

The AD app may be available in a plurality of languages, such as German and English. The user can change the app's language in Settings. The AD app should start with the default language from the respective computing device operating software (e.g., IOS for Apple IPHONE, Android for Samsung Galaxy smartphones, Google Pixel smartphones, and the like). If that language is not supported by the AD app, the AD app language may change to English.

The AD app may be programmed with an open, internationally recognized interface and semantic standards for interoperable e-health infrastructure.

The AD app may allow the user to export therapy-relevant extracts of the collected data in human-readable and printable form (e.g. PDF) in order to use them for their own purposes or pass them on to a doctor.

The AD app may offer operating aids for users with restricted access or support the existing operating aids.

The usability guidelines of the respective platform for mobile applications may be fully implemented with respect to the AD app.

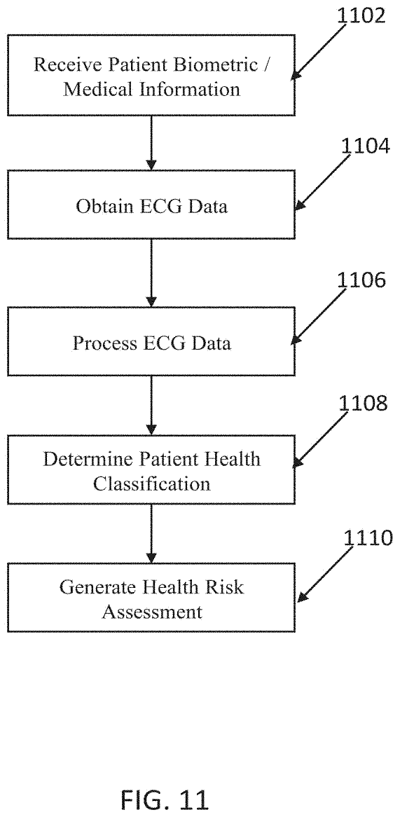

FIG. 11 provides an exemplary flow chart for a method described herein for generating a health risk assessment. The AD app may first receive ECG data 1102 from a corresponding ECG recording. The ECG recording may be measured via an ECG device. As described herein, in some cases, the ECG device is provided with a wearable device. In such cases, the ECG data may be communicated to an ECG app with the wearable device, and relayed to an AD App via a cloud server (for example). In some cases the AD App receives the ECG data directly from the ECG device. In some cases, the ECG device is located at a health care facility. In some cases, the ECG data is obtained from a prior ECG recording (for example, from a health care facility or wearable device), which may be provided as a PDF file (e.g., as an image), and which may be provided to the AD app via a user interface and/or a cloud server (for example).

The obtained ECG data may be in ECG raw data form, as described herein. The ECG raw data may then optionally be processed 1104, for example, outputting R-R intervals as a column chart, as well as secondary characteristics as a graphical output (for example see FIGS. 3-7, and 13-15). The AD app and/or the ECG app, as described herein, or another computing device, may be configured to process the ECG raw data. The AD app may then proceed to characterize the ECG data 1106 based on the analysis, such as determining the heart rhythm and identifying other characteristics of the ECG data. The AD may characterize the ECG data based on the ECG raw data, the secondary characteristics of the ECG data, the R-R intervals, and/or using summary statistics of the R-R intervals (see for example FIGS. 3-7 and 13-15). As described herein, the AD app may use AI (with a machine learning algorithm) to characterize the ECG data, based on a comparison of trained data sets (such as reference pools as described herein). As described herein, the terms “AI” or “Artificial Intelligence” may be used interchangeably with the term “classifier”. In some cases, the ECG data (ECG recording) is provided to a machine learning algorithm, via a cloud server (for example), wherein the machine learning algorithm will compare against a plurality of trained data sets to characterize the ECG data. The patient health characterization is then relayed back to the AD App. In some cases, the machine learning algorithm is provided with the AD App, which accesses the trained data sets (via a cloud server for example), to characterize the patient information.

In some cases, the other characteristics of the ECG data identified by the AD app include identifying ECG abnormalities, which may include QRS fragmentation. QRS fragmentation reflects a conduction disturbance during electrical activation of the heart ventricles and occurs predominantly in patients with coronary artery disease or what is called “non-ischemic (dilated) cardiomyopathy”. In these patients, the QRS complexes all do not just show a single Q wave, a single R wave, and a single S wave but have one or more additional “spikes” apparent on the R-wave upstroke or downstroke or the S-wave downstroke. This is called “fragmentation” and it may be present in narrow as well as wide QRS complexes and in sinus rhythm as well as during atrial fibrillation.

Optionally, in some case, the AD app receives patient biometric/medical information (not shown).

The AD app may optionally then determine a patient health classification 1108 (for example, designate a Atrial Fibrillation group (AF Group), as described herein) by using the ECG characterization.

Based on the ECG characterization and/or the patient health classification, a health risk assessment is generated 1110 via the AD App, and output to the patient. Examples of the health risk assessment, as described herein, include a stroke risk determination and a sudden cardiac death determination. The health risk assessment may be output using an AD app, as described herein.

Optionally, the AD app may further receive biometric information and other patient information which may be used by the AD app in the determining a patient health classification 1108 and/or generating a health risk assessment 1110. The biometric information and other patient information may include gender, age, sex, current symptoms, smoking status, eating habits, physical exercise frequency, etc., or any combination thereof.

### Computer Systems

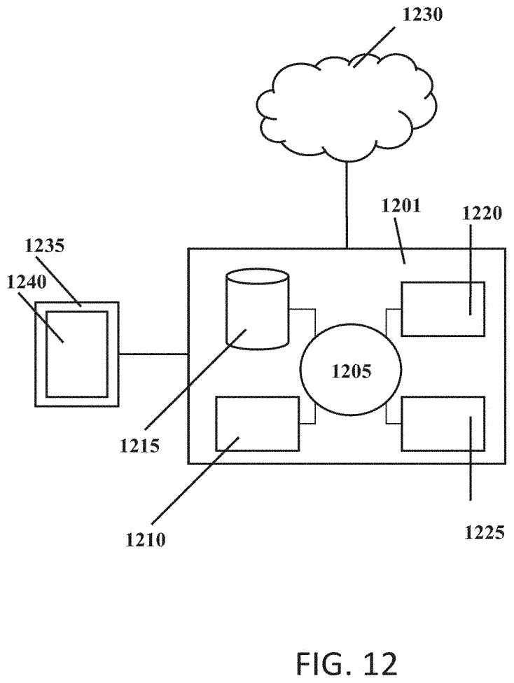

The present disclosure provides computer systems that are programmed to implement methods of the disclosure, including the control of the multi-detection system, control hardware components, receive and process data, interface with a user, etc. FIG. 12 shows a computer system 1201 that is programmed or otherwise configured to operate and/or control the data module and the processing module. The computer system 1201 can regulate various aspects of the present disclosure, such as, for example, determining neurological conditions, classification of neurological conditions, classification of EEG signals, classification of seizures, classification of delirium, classification of stroke, classification of sedation, generate notifications, generate probability plots of neurological conditions, processing EEG signals, segmenting EEG signals, extracting features, processing features with machine learning algorithms, relating features to assessment scales, implementing the control policy and neurological condition burden, calculating the value, plotting the probability of the neurological conditions, etc. The computer system 1201 can be an electronic device of a user or a computer system that is remotely located with respect to the electronic device. The electronic device can be a mobile electronic device.

The computer system 1201 includes a central processing unit (CPU, also “processor” and “computer processor” herein) 1205, which can be a single core or multi core processor, or a plurality of processors for parallel processing. The computer system 1201 also includes memory or memory location 1210 (e.g., random-access memory, read-only memory, flash memory), electronic storage unit 1215 (e.g., hard disk), communication interface 1220 (e.g., network adapter) for communicating with one or more other systems, and peripheral devices 1225, such as cache, other memory, data storage and/or electronic display adapters. The memory 1210, storage unit 1215, interface 1220 and peripheral devices 1225 are in communication with the CPU 1205 through a communication bus (solid lines), such as a motherboard. The storage unit 1215 can be a data storage unit (or data repository) for storing data. The computer system 1201 can be operatively coupled to a computer network (“network”) 1230 with the aid of the communication interface 1220. The network 1230 can be the Internet, an internet and/or extranet, or an intranet and/or extranet that is in communication with the Internet. The network 1230 in some cases is a telecommunication and/or data network. The network 1230 can include one or more computer servers, which can enable distributed computing, such as cloud computing. The network 1230, in some cases with the aid of the computer system 1201, can implement a peer-to-peer network, which may enable devices coupled to the computer system 1201 to behave as a client or a server.

The CPU 1205 can execute a sequence of machine-readable instructions, which can be embodied in a program or software. The instructions may be stored in a memory location, such as the memory 1210. The instructions can be directed to the CPU 1205, which can subsequently program or otherwise configure the CPU 1205 to implement methods of the present disclosure. Examples of operations performed by the CPU 1205 can include fetch, decode, execute, and writeback.

The CPU 1205 can be part of a circuit, such as an integrated circuit. One or more other components of the system 1201 can be included in the circuit. In some cases, the circuit is an application specific integrated circuit (ASIC).

The storage unit 1215 can store files, such as drivers, libraries and saved programs. The storage unit 1215 can store user data, e.g., user preferences and user programs. The computer system 1201 in some cases can include one or more additional data storage units that are external to the computer system 1201, such as located on a remote server that is in communication with the computer system 1201 through an intranet or the Internet.

The computer system 1201 can communicate with one or more remote computer systems through the network 1230. For instance, the computer system 1201 can communicate with a remote computer system of a user (e.g., neurological condition detection system manager, neurological condition detection system user, neurological condition data acquirer, neurological condition detection system scribe). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1201 via the network 1230.

Methods as described herein can be implemented by way of machine (e.g., computer processor) executable code stored on an electronic storage location of the computer system 1201, such as, for example, on the memory 1210 or electronic storage unit 1215. The machine executable or machine readable code can be provided in the form of software. During use, the code can be executed by the processor 1205. In some cases, the code can be retrieved from the storage unit 1215 and stored on the memory 1210 for ready access by the processor 1205. In some situations, the electronic storage unit 1215 can be precluded, and machine-executable instructions are stored on memory 1210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1201 can include or be in communication with an electronic display 1235 that comprises a user interface (UI) 1240 for providing, for example, a login screen for an administrator to access software programmed to control the multi-indication detection system and functionality and/or for providing the operation status health of the multi-indication detection system. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1205. The algorithm can, for example, be component of software described elsewhere herein and may modulate the seizure detection system parameters (e.g. processing EEG signals, machine learning algorithms, control policy, neurological condition burden, notifications, etc.).

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “patient” and “subject” may be used interchangeably herein.

As used herein, the term “about” a number refers to that number plus or minus 10% of that number. The term “about” a range refers to that range minus 10% of its lowest value and plus 10% of its greatest value.

As used herein, the term “AI” refers to artificial intelligence. In some cases, AI may include the use of a machine learning algorithm. The term “AI” and “artificial intelligence” may be used interchangeably with the term “classifier”.

As used herein, the term “ECG recording” may refer to a measurement of an ECG using an ECG device, as described herein. In some cases, the ECG recording comprises ECG data, which may include ECG raw data and/or processed ECG data.

As used herein, the term “ECG raw data” refers to raw, unprocessed ECG data measured (for example, the data that make up the graphical output in FIG. 13A).

As used herein, the term “processed ECG data” refers to processed ECG raw data, which may include outputting secondary ECG characteristics (for example, see FIG. 13B), and/or outputting R-R interval graphs (for example, see FIG. 13C).

As used herein, the term “R-R” refers to a duration between heartbeats (which may be measured in milliseconds). The term “R-R” and “RR” may be used interchangeably.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. (canceled)

2. A method for health risk assessment and monitoring for a user, the method comprising:
(a) collecting a first set of user data comprising one or more of biometric information or medical information relating to the user;
(b) receiving ECG data for the user measured during a first evaluation period;
(c) identifying irregularities in the received ECG data for the user measured during the first evaluation period by applying a machine learning algorithm to said ECG data;
(d) classifying the user into a stroke risk group among a plurality of stroke risk groups based on the identified irregularities, the plurality of stroke risk groups comprises an SR risk group, an AF I risk group, an AF II risk group, and an AF III risk group, and wherein classifying the user comprises:
(i) classifying the user into the SR risk group if no irregularities are identified during the first evaluation period and receiving further ECG data measured from the user at a first measurement frequency during a second evaluation period,
(ii) classifying the user into an AF I risk group if an irregularity is identified during the first evaluation period and receiving further ECG data measured from the user at a second measurement frequency during the second evaluation period, the second measurement frequency being higher than the first measurement frequency, and repeating step (c) for the further ECG data measured during the second evaluation period,
(iii) classifying the user into an AF II risk group if an irregularity is identified from the further ECG data during the second evaluation period and receiving further ECG data measured from the user at a third measurement frequency during a third evaluation period longer than the second evaluation period, the third measurement frequency being lower than the second measurement frequency, and repeating step (c) for the further ECG data measured during the third evaluation period, and
(iv) classifying the user into an AF III risk group if an irregularity is identified from the further ECG data during the third evaluation period and receiving further ECG data measured from the user at a fourth measurement frequency during a fourth evaluation period longer than the third evaluation period;
(e) determining a risk of sudden cardiac death of the user based on the user data and a frequency of irregularities identified in the ECG data received during one or more of the evaluation periods;
(f) determining a 10-year risk of a cardiovascular event of the user based the user data and a Systematic Coronary Risk Estimation score of the user;
(g) generating a set of recommendations for the user based on (i) the stroke risk group of the user, (ii) the risk of sudden cardiac death, and (iii) the 10-year risk of a cardiovascular event.

3. The method of claim 2, wherein the biometric information or medical information comprises one or more of height, weight, waist circumference, smoking status, country of residence, total cholesterol, LDL cholesterol, systolic blood pressure, medication, congestive heart failure, ischemic cardiomyopathy, nonischemic cardiomyopathy, NYHA functional class, LV ejection fraction, hypertension, diabetes mellitus, glycated hemoglobin, vascular disease, prior TIA/stroke/thromboembolism, prior myocardial infarction, valvular heart disease, pacemaker, syncope, or a combination thereof.

4. The method of claim 2, further comprises steps for updating the health risk assessment of the user:
(h) collecting an updated set of user data comprising updated biometric information or updated medical information;
(i) receiving further ECG data measured from the user after the fourth evaluation period, and repeating step (c) for the further ECG data measured after the fourth evaluation period;
(j) classifying the user into an updated stroke risk group based on the stroke risk group of the user and the updated set of user data;
(k) determining an updated risk of sudden cardiac death of the user based on the risk of sudden cardiac death and the updated set of user data and a frequency of irregularities identified in the ECGs received after the fourth evaluation period;
(l) determining an updated 10-year risk of a cardiovascular event of the user based the 10-year risk of a cardiovascular event, the updated set of user data, and an updated Systematic Coronary Risk Estimation score for the user;
(m) generating an updated set of recommendations for the user based on (i) the updated stroke risk group of the user, (ii) the updated risk of sudden cardiac death, and (iii) the updated 10-year risk of a cardiovascular event.

5. The method of claim 4, wherein the ECG data is received at a minimum frequency after the fourth evaluation period.

6. The method of claim 4, wherein steps (h)-(m) are repeated after a threshold period of time or continuously.

7. The method of a claim 2, wherein identifying irregularities in the received ECG data comprises:
(a) creating a graphical output based on the ECG data;
(b) determining a statistical computation corresponding to the graphical output; and
(c) comparing the statistical computation with a reference statistical data so as to (i) identify ECG data corresponding to sinus rhythm, (ii) identify ECG data corresponding to a type of cardiac arrhythmia, or (iii) a combination thereof.

8. The method of claim 7, wherein analyzing the received ECG data comprises performing on the received ECG data (1) mean and standard deviation of the R-R intervals, (2) the median (e.g., 50th percentile) of the R-R intervals, (3) 25th and 75th percentile of the R-R intervals, (4) root mean square of successive differences between R-R intervals, or (5) any combination thereof.

9. The method of claim 7, wherein the reference statistical data corresponds to a plurality of sets of reference ECG data, wherein the plurality of sets of reference ECG data are obtained from a plurality of other users, wherein each set of reference ECG data of the plurality of set of reference ECG data comprises reference R-R intervals corresponding to sinus rhythm and a corresponding mean heart rate range, wherein each set of reference ECG data comprises at least 50, 100, 200, 500, or 1000 electrocardiograms corresponding to sinus rhythm.

10. The method of claim 2, wherein the sudden cardiac death risk of the user is determined based on a presence and/or absence of fractionated QRS complexes identified with the received ECG data.

11. A computer-implemented system comprising:
a processor,
a memory coupled to the processor and storing instructions for the processor to generate a health risk assessment for the user according to the method of any one of claims 2-10.
